(19) United States
(12) Patent Application Publication
(10) Pub. No.: US 2025/0074899 A1
(43) Pub. Date: Mar. 6, 2025

(54) A2-ADRENOCEPTOR SUBTYPE C ANTAGONISTS FOR THE TREATMENT OF SLEEP APNEA

(71) Applicant: Bayer Aktiengesellschaft, Leverkusen (DE)

(72) Inventors: Martina DELBECK, Heiligenhaus (DE); Michael HAHN, Langenfeld (DE)

(21) Appl. No.: 18/726,361

(22) PCT Filed: Jan. 5, 2023
(86) PCT No.: PCT/EP2023/050164

§ 371 Date: Jul. 2, 2024

(30) Foreign Application Priority Data
Jan. 7, 2022 (EP) 22150561.3

(51) Int. Cl. C07D 405/04; A61K 31/4433; A61K 31/453; A61K 45/06; A61P 11/00

(52) U.S. Cl. CPC C07D 405/04; A61K 31/4433; A61K 31/453; A61K 45/06; A61P 11/00

(57) ABSTRACT

The present invention relates to α2-Adrenoceptor subtype C (alpha-2C) antagonists, in particular 3-substituted 1-(2,3-dihydrobenzo[1,4]dioxin-2-ylmethyl)azacycles of formula (I) for the use in a method for the treatment and/or prophylaxis of sleep-related breathing disorders, preferably obstructive and central sleep apneas and snoring.

The present invention relates to α2-Adrenoceptor subtype C (alpha-2C) antagonists, in particular 3-substituted 1-(2,3-dihydrobenzo[1,4]dioxin-2-ylmethyl)azacycles of formula (I) for the use in a method for the treatment and/or prophylaxis of sleep-related breathing disorders, preferably obstructive and central sleep apneas and snoring.

### BACKGROUND OF THE INVENTION

Obstructive sleep apnoea (OSA) is a sleep-related respiratory disorder which is characterized by repeat episodes of obstruction of the upper airways. When breathing in, the patency of the upper airways is ensured by the interaction of two opposite forces. The dilative effects of the musculature of the upper airways counteract the negative intraluminal pressure, which constricts the lumen. The active contraction of the diaphragm and the other auxiliary respiratory muscles generates a negative pressure in the airways, thus constituting the driving force for breathing. The stability of the upper respiratory tract is substantially determined by the coordination and contraction property of the dilating muscles of the upper airways.

Upper airway collapse in OSA is thought to occur at sleep onset because of the reduction of activity of several upper airway dilator muscles, which as a consequence are unable to maintain the anatomically vulnerable airway open. However, some upper airway dilator muscles, including the genioglossus muscle, which is the most important of the dilating muscles of the upper respiratory airway and which is innervated by the hypoglossal nerve, can increase activity during sleep in response to respiratory stimuli, potentially counteracting some of these changes at sleep onset. It was observed that OSA patients have apnea free intervals in which the genioglossus muscle activity is only 25-40% higher compared with sleep phases with frequent obstructive apneas (Jordan A S, White D P, Lo Y L et al., Airway dilator muscle activity and lung volume during stable breathing in obstructive sleep apnea. Sleep 2009, 32(3): 361-8). Noradrenaline is one of the most potent neuromodulators of hypoglossal motoneuron activity (Horner R. L. Neuromodulation of hypoglossal motoneurons during sleep. Respir Physiol Neurobiol 2008, 164 (1-2): 179-196). It is thought, that decreased noradrenergic drive leads to sleep-dependent decline of hypoglossal motoneuron excitability resulting in reduced upper airway dilator muscle activity, especially reduced genioglossus muscle activity.

Alpha2C adrenoceptors regulate the release of noradrenaline from central noradrenergic neurons, they are autoreceptors involved in presynaptic feedback inhibition of noradrenaline (Hein L. et al, Two functionally distinct alpha2-adrenergic receptors regulate sympathetic neurotransmission Nature 1999, 402(6758): 181-184). An increase in the activity of the motoneurons of the hypoglossal nerve through Alpha2c adrenoceptor antagonism can stabilize the upper airways and protect them from collapse and occlusion. Moreover, also snoring can be inhibited through the mechanism of stabilization of the upper respiratory airways.

For simple snoring, there is no obstruction of the upper airways. By the narrowing of the upper airways, the flow velocity of the inhaled and exhaled air increases. This together with the relaxed muscles causes fluttering of the soft tissues of the mouth and throat in the airflow. This slight vibration generated the typical snoring sounds.

Obstructive snoring (upper airway resistance syndrome, heavy snoring, hypopnea syndrome) is caused by a recurrent partial obstruction of the upper airway during sleep. This results in an increase in airway resistance and thus to an increase in work of breathing with significant intrathoracic pressure fluctuations. The negative intrathoracic pressure development during inspiration can thereby reach values as they occur as a result of a complete airway obstruction in OSA. The pathophysiological effects on the heart, circulation and sleep quality are the same as in obstructive sleep apnea. The pathogenesis is likely the same as in OSA. Obstructive snoring often provides the precursor for OSA (Hollandt J. H. et al., Upper airway resistance syndrome (UARS)-obstructive snoring. HNO 2000, 48(8): 628-634).

Central sleep apnea (CSA) occurs as a result of disturbed brain function or impaired respiratory regulation. CSA is characterized by a lack of drive to breathe during sleep, resulting in repetitive periods of insufficient or absent ventilation and compromised gas exchange. There are several manifestations of CSA. These include high altitude-induced periodic breathing, idiopathic CSA (ICSA), narcotic-induced central apnea, obesity hypoventilation syndrome (OHS), and Cheyne-Stokes breathing (CSB). While the precise precipitating mechanisms involved in the various types of CSA may vary considerably, unstable ventilatory drive during sleep is a principal underlying feature (Eckert D. J. et al., Central sleep apnea: Pathophysiology and treatment. Chest 2007, 131(2): 595-607).

US 2018/0235934 A1 describes methods for treating disorders such as obstructive sleep apnea using agents for promoting hypoglossal motoneuron excitability. As agents for promoting hypoglossal motoneuron excitability a disinhibtor and/or stimulant of central noradrenic neurons is described. In some embodiments the disinhibitor of central noradrenergic neurons is an alpha2-adrenoceptor antagonist such as yohimbine or an alpha2-adrenoceptor subtype A (alpha-2A) antagonists or alpha2-adrenoceptor subtype C (alpha-2C) antagonist. The alpha2-adrenoceptor antagonist are selected from the group consisting of Atipamezole, MK-912, RS-79948, RX 821002, [3H]2-methoxy-idazoxan and JP-1302.

Alpha2C adrenoceptors belong to the family of G-protein coupled receptors. Beside the different Alpha1-adrenoceptors three different Alpha2-adrenoceptor subtypes exist (Alpha2A, Alpha2B and Alpha2C). They are involved in the mediation of several diverse physiologic effects in different tissues upon stimulation by endogeneous catecholamines (epinephrine, norepinephrine), either derived from synapses or via the blood. Alpha2 adrenoceptors plays an important physiological role, mainly in the cardiovascular system and in the central nervous system. Alpha2A- and Alpha2C-adrenoceptors are the main autoreceptors involved in presynaptic feedback inhibition of noradrenaline in the central nervous system. The potency and affinity of noradrenaline at the Alpha2C-adrenoceptor is higher than that for the Alpha2A-adrenoceptor. The Alpha2C-adrenoceptor inhibits noradrenaline release at low endogenous concentrations of noradrenaline, while Alpha2A-adrenoceptors inhibit noradrenaline release at high endogenous noradrenaline concentrations (Uys M. M. et al. Therapeutic Potential of Selectively Targeting the α2C-Adrenoceptor in Cognition, Depression, and Schizophrenia—New Developments and Future Perspective. Frontiers in Psychiatry 2017 Aug. 14; 8:144. doi: 10.3389/fpsyt.2017.00144. eCollection 2017).

Aryl piperazines as α2-Adrenoceptor subtype C (alpha-2C) antagonists as well as their preparation and the use thereof as a medicament are known from WO 03/082866 A1 where the compounds are disclosed as useful for the treatment for disorders such as disorder propagated by stress, Parkinson's disease, depression, schizophrenia, attention deficit hyperactivity disorder, post-traumatic stress disorder, obsessive compulsive disorder, Tourette's syndrome, blepharospasm or other focal dystonias, temporal lobe epilepsy with psychosis, a drug-induced psychosis, Huntington's disease, a disorder caused by fluctuation of the levels of sex hormones, panic disorder, Alzheimer's disease or mild cognitive impairment. There is nothing disclosed about the use of these compounds in the treatment of sleep-related breathing disorders, preferably obstructive and central sleep apneas and snoring.

The current gold standard treatment for patients with OSA is continuous positive airway pressure (CPAP). The positive airflow pressure that is generated by an airflow turbine pump splints open the upper airway, reversing all potential causes of pharyngeal collapse, thereby preventing hypopneas, apneas and sleep fragmentation. Unfortunately, up to 50% of all patients with OSA do not tolerate CPAP in the long-term (M. Kohler, D. Smith, V. Tippett et al., Thorax 2010 65(9):829-32: Predictors of long-term compliance with continuous positive airway pressure). Therefore, there is still the need to find effective therapeutic agents for the treatment and/or prophalxis of sleep-related breathing disorders such as obstructive sleep apnea. Therefore the object of the present invention is to provide an effective therapeutic agent for the treatment and/or prophalxis of sleep-related breathing disorders, for example of obstructive sleep apnea, central sleep apnea and snoring.

Surprisingly, it has now been found that 3-substituted 1-(2,3-dihydrobenzo[1,4]dioxin-2-ylmethyl)azacycles of formula (I) of the present invention inhibit upper airway collapsibility and are thus suitable for the production of medicaments for the use in the treatment and/or prophylaxis of sleep-related breathing disorders, preferably obstructive and central sleep apneas and snoring.

The present invention relates to compounds of formula (I)

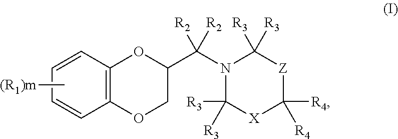

wherein
X is C(R₅)(R₆) or C(R₇)(R₈);
Z is —[C(R₄)₂]ₙ— or a single bond;
R₁ is, independently at each occurrence, hydroxy, (C₁-C₆)alkyl, (C₁-C₆)alkoxy, halogen, halo(C₁-C₆)alkyl, phenyl(C₁-C₆)alkoxy, (C₁-C₆)alkoxy-(C═O)—, CN, NO₂, NH₂, mono- or di(C₁-C₆)alkylamino or carboxy;
R₂ is, independently at each occurrence, H or (C₁-C₆)alkyl;
R₃ is, independently at each occurrence, H or (C₁-C₆)alkyl;
R₄ is, independently at each occurrence, H, hydroxy, (C₁-C₆)alkyl, (C₂-C₆)alkenyl, hydroxy(C₁-C₆)alkyl, hydroxy(C₂-C₆)alkenyl, (C₁-C₆)alkoxy(C₁-C₆)alkyl, (C₂-C₆)alkenyloxy(C₁-C₆)alkyl, (C₁-C₆)alkyl-(C═O)—, (C₁-C₆)alkoxy-(C═O)—, hydroxy(C₁-C₆)alkoxy(C₁-C₆)alkyl or (C₁-C₆)alkoxy(C₁-C₆)alkoxy(C₁-C₆)alkyl;
or
R₄ and R₄ both attached to the same carbon ring atom form, together with the carbon ring atom to which they are attached, a —(C═O)— group;
R₅ is H or hydroxy;
or
R₄ and R₅ attached to adjacent carbon ring atoms form a bond between the carbon ring atoms to which they are attached;
R₆ is phenyl unsubstituted or substituted with 1 or 2 substituent(s) R₉;
R₇ is (C₁-C₆)alkyl, hydroxy(C₁-C₆)alkyl, (C₁-C₆)alkoxy(C₁-C₆)alkyl or (C₁-C₆)alkoxy-(C═O)—;
R₈ is hydroxy, (C₁-C₆)alkyl, (C₂-C₆)alkenyl, (C₁-C₆)alkoxy, halogen, hydroxy(C₁-C₆)alkyl, halo(C₁-C₆)alkyl, hydroxy(C₂-C₆)alkenyl, hydroxy(C₁-C₆)alkoxy, halo(C₁-C₆)alkoxy, phenyl(C₁-C₆)alkoxy, (C₁-C₆)alkoxy(C₁-C₆)alkyl, (C₂-C₆)alkenyloxy(C₁-C₆)alkyl, (C₁-C₆)alkyl-(C═O)—, (C₁,—C₆)alkoxy-(C═O)—, (C₁-C₆)alkoxy-(C═O)—(C₁-C₆)alkyl, (C₁-C₆)alkyl-S—, (C₁-C₆)alkyl-(C═O)—O—, (C₁-C₆)alkyl-(C═O)—O—(C₁-C₆)alkyl, hydroxy-(C═O)—(C₁-C₆)alkoxy, hydroxy-(C═O)—(C₁-C₆)alkoxy(C₁-C₆)alkyl, (C₁-C₆)alkoxy-(C═O)—(C₁-C₆)alkoxy(C₁-C₆)alkyl, (C₁-C₆)alkyl-S—(C₁-C₆)alkyl, hydroxy(C₁-C₆)alkyl-S—(C₁-C₆)alkyl, (C₁-C₆)alkoxy(C₁-C₆)alkyl-S—(C₁-C₆)alkyl, (C₁-C₆)alkyl-(C═O)—S—(C₁-C₆)alkyl (C₁-C₆)alkoxy-(C═—O)—(C₁-C₆)alkyl-S—(C₁-C₆)alkyl, (C₁-C₆)alkyl-S—(C₁-C₆)alkoxy(C₁-C₆)alkyl, hydroxy(C₁-C₆)alkyl-S—(C₁-C₆)alkoxy(C₁-C₆)alkyl, (C₁-C₆)alkoxy(C₁-C₆)alkyl-S—(C₁-C₆)alkoxy(C₁-C₆)alkyl, (C₁-C₆)alkyl-(O═S═O)—(C₁-C₆)alkyl, hydroxy(C₁-C₆)alkyl-(O═S═O)—(C₁-C₆)alkyl, (C₁-C₆)alkoxy(C₁-C₆)alkyl-(O═S═O)—(C₁-C₆)alky, (C₁-C₆)alkyl-(O═S═O)—(C₁-C₆)alkoxy(C₁-C₆)alkyl, hydroxy(C₁-C₆)alkyl-(O═S═O)—(C₁-C₆)alkoxy(C₁-C₆)alkyl, (C₁-C₆)alkoxy(C₁-C₆)alkyl-(O═S═O)—(C₁-C₆)alkoxy(C₁-C₆)alkyl, (C₁-C₆)alkyl-O—(O═S═O)—(C₁-C₆)alkyl, hydroxy(C₁-C₆)alkyl-O—(O═S═O)—(C₁-C₆)alkyl, (C₁-C₆)alkyl-(O═S═O)—O—(C₁-C₆)alkyl, hydroxy(C₁-C₆)alkyl-(O═S═O)—O—(C₁-C₆)alkyl, (C₁-C₆)alkyl-(O═S═O)—N(R₁₀)—(C₁-C₆)alkyl, hydroxy(C₁-C₆)alkoxy(C₁-C₆)alkyl, (C₁-C₆)alkoxy(C₁-C₆)alkoxy(C₁-C₆)alkyl, halo(C₁-C₆)alkoxy(C₁-C₆)alkyl, CN, NO₂, (R₁₀)₂N—, (R₁₀)₂N—(C₁-C₆)alkyl, (R₁₀)₂N—(C═O)—, (R₁₀)₂N—(C═O)—(C₁-C₆)alkoxy(C₁-C₆)alkyl, (R₁₀)₂N—(C═O)—(C₁-C₆)alkyl-S—(C₁-C₆)alkyl, (R₁₀)₂N—(C═O)—(C₁-C₆)alkyl-(O═S═O)—(C₁-C₆)alkyl, carboxy, R₁₁—(O═S═O)—, R₁₁—(O═S═O)—O— or (C₁-C₆)alkoxy-(C₁-C₆)alkoxy;
or
R₄ and R₈ attached to adjacent carbon ring atoms form, together with the carbon ring atoms to which they are attached, a condensed phenyl ring, wherein said phenyl ring is unsubstituted or substituted with 1 or 2 substituent(s) each independently being hydroxy, (C₁-C₆)alkyl, (C₁-C₆)alkoxy, hydroxy(C₁-C₆)alkyl, halo(C₁-C₆)alkyl, (C₁-C₆)alkoxy(C₁-C₆)alkyl, (C₁-C₆)alkyl-(C═O)— or (C₁-C₆)alkoxy-(C═O)—;
or
R₄ and R₈ attached to adjacent carbon ring atoms form, together with the carbon ring atoms to which they are attached, a condensed 5 or 6 membered saturated or unsaturated carbocyclic ring or a condensed 5 or 6 membered saturated or unsaturated heterocyclic ring containing 1 or 2 heteroatom(s) selected from N, O, and S,
   wherein said
   carbocyclic or heterocyclic ring is unsubstituted or substituted with 1 or 2 substituent(s) each independently being hydroxy, (C₁-C₆)alkyl, (C₁-C₆)alkoxy, hydroxy(C₁-C₆)alkyl, halo(C-C₆)alkyl, (C₁-C₆)alkoxy(C₁-C₆)alkyl, (C₁-C₆)alkyl-(C═O)—, (C1-C6)alkoxy-(C═O)— or oxo;
or
R₇ and R₈ form, together with the carbon ring atom to which they are attached, a 5 or 6 membered saturated carbocyclic ring or a 5 or 6 membered saturated heterocyclic ring containing 1 or 2 heteroatom(s) selected from N, O, and S, wherein said carbocyclic or heterocyclic ring is unsubstituted or substituted with 1 or 2 substituent(s) each independently being hydroxy, (C₁-C₆)alkyl or oxo;
R₉ is, independently at each occurrence, hydroxy, (C₁-C₆)alkyl, (C₂-C₆)alkenyl, (C₁-C₆)alkoxy, halogen, hydroxy(C₁-C₆)alkyl, halo(C₁-C₆)alkyl, hydroxy(C₂-C₆)alkenyl, hydroxy(C₁-C₆)alkoxy, halo(C₁-C₆)alkoxy, phenyl(C₁-C₆)alkoxy, (C₁-C₆)alkoxy(C₁-C₆)alkyl, (C₂-C₆)alkenyloxy(C₁-C₆)alkyl, (C₁-C₆)alkyl-(C═O)—, (C₁-C₆)alkoxy-(C═O)—, (C₁-C₆)alkoxy-(C═O)—(C₁-C₆)alkyl, (C₁-C₆)alkyl-S—, (C₁-C₆)alkyl-(C═O)—O—, (C₁-C₆)alkyl-(C═O)—O—(C₁-C₆)alkyl, hydroxy-(C═O)—(C₁-C₆)alkoxy, hydroxy-(C═O)—(C₁-C₆)alkoxy(C₁-C₆)alkyl, (C₁-C₆)alkoxy-(C═O)—(C₁-C₆)alkoxy(C₁-C₆)alkyl, (C₁-C₆)alkyl-S—(C₁-C₆)alkyl, hydroxy(C₁-C₆)alkyl-S—(C₁-C₆)alkyl, (C₁-C₆)alkoxy(C₁-C₆)alkyl-S—(C₁-C₆)alkyl, (C₁-C₆)alkyl-(C═O)—S—(C₁-C₆)alkyl, (C₁-C₆)alkoxy-(C═O)—(C₁-C₆)alkyl-S—(C₁-C₆)alkyl, (C₁-C₆)alkyl-S—(C₁-C₆)alkoxy(C₁-C₆)alkyl, hydroxy(C₁-C₆)alkyl-S—(C₁-C₆)alkoxy(C₁-C₆)alkyl, (C₁-C₆)alkoxy(C₁-C₆)alkyl-S—(C₁-C₆)alkoxy(C₁-C₆)alkyl, (C₁-C₆)alkyl-(O═S═O)—(C₁-C₆)alkyl, hydroxy(C₁-C₆)alkyl-(O═S═O)—(C₁-C₆)alkyl, (C₁-C₆)alkoxy(C₁-C₆)alkyl-(O═S═O)—(C₁-C₆)alkyl, (C₁-C₆)alkyl-(O═S═O)—(C₁-C₆)alkoxy(C₁-C₆)alkyl, hydroxy(C₁-C₆)alkyl-(O═S═O)—(C₁-C₆)alkoxy(C₁-C₆)alkyl, (C₁-C₆)alkoxy(C₁-C₆)alkyl-(O═S═O)—(C₁-C₆)alkoxy(C₁-C₆)alkyl, (C₁-C₆)alkyl-O—(O═S═O)—(C₁-C₆)alkyl, hydroxy(C₁-C₆)alkyl-O—(O═S═O)—(C₁-C₆)alkyl, (C₁-C₆)alkyl-(O═S═O)—O—(C₁-C₆)alkyl, hydroxy(C₁-C₆)alkyl-(O═S═O)—O—(C₁-C₆)alkyl, (C₁-C₆)alkyl-(O═S═O)—N(R₁₀)—(C₁-C₆)alkyl, hydroxy(C₁-C₆)alkoxy(C₁-C₆)alkyl, (C₁-C₆)alkoxy(C₁-C₆)alkoxy(C₁-C₆)alkyl, halo(C₁-C₆)alkoxy(C₁-C₆)alkyl, CN, NO₂, (R₁₀)₂N—, (R₁₀)₂N—(C₁-C₆)alkyl, (R₁₀)₂N—(C═O)—, (R₁₀)₂N—(C═O)—(C₁-C₆)alkoxy(C₁-C₆)alkyl, (R₁₀)₂N—(C═O)—(C₁-C₆)alkyl-S—(C₁-C₆)alkyl, (R₁₀)₂N—(C═O)—(C₁-C₆)alkyl-(O═S═O)—(C₁-C₆)alkyl, carboxy, R₁₁—(O═S═O)— or R₁₁—(O═S═O)—O—;
or
R₉ and R₉ attached to adjacent carbon ring atoms form, together with the carbon ring atoms to which they are attached, a condensed phenyl ring, a condensed 5 or 6 membered unsaturated carbocyclic ring or a condensed 5 or 6 membered unsaturated heterocyclic ring containing 1 or 2 heteroatom(s) selected from O and S, wherein said phenyl, carbocyclic or heterocyclic ring is unsubstituted or substituted with 1 or 2 substituent(s) each independently being hydroxy, (C₁-C₆)alkyl, (C₁-C₆)alkoxy, hydroxy(C₁-C₆)alkyl, halo(C₁-C₆)alkyl, (C₁-C₆)alkoxy(C₁-C₆)alkyl, (C₁-C₆)alkyl-(C═O)— or (C₁-C₆)alkoxy-(C═O)—;
R₁₀ is, independently at each occurrence, H, (C₁-C₆)alkyl, hydroxy(C₁-C₆)alkyl, (C₁-C₆)alkyl-(C═O)— or R₁₁—(O═S═O)—;
R₁₁ is, independently at each occurrence, hydroxy, (C₁-C₆)alkyl, (C₁-C₆)alkoxy, halo(C₁-C₆)alkyl or mono- or di(C₁-C₆)alkylamino;
m is 0, 1 or 2; and
n is 1 or 2;
or a pharmaceutically acceptable salt or ester thereof,
with the provisos that the compound is not 1-(7-tert-butyl-2,3-dihydrobenzo[1,4]dioxin-2-ylmethyl)-3,3-dimethyl-piperidine, 1-(6-tert-butyl-2,3-dihydrobenzo[1,4]dioxin-2-ylmethyl)-3,3-dimethyl-piperidine, 2-(2,3-dihydrobenzo[1,4]dioxin-2-ylmethyl)-6,7-dimethoxy-1-methyl-1,2,3,4-tetrahydro-isoquinoline, 2-(2,3-dihydrobenzo[1,4]dioxin-2-ylmethyl)-6,7-dimethoxy-1,2,3,4-tetrahydro-isoquinoline, 2-(2,3-dihydrobenzo[1,4]dioxin-2-ylmethyl)-1,2,3,4-tetrahydroisoquinoline or 1-(2,3-dihydro-benzo[1,4]dioxin-2-ylmethyl-3-(3-trifluoromethyl-phenyl)-pyrrolidine,
for the use in a method for the treatment and/or prophylaxis of sleep-related breathing disorders,
preferably obstructive and central sleep apneas and snoring.

In a possible subgroup of the compounds of formula I, Z is —[C(R₄)₂]n-.

In a further possible subgroup of the compounds of formula I, n is 1.

In another possible subgroup of the compounds of formula I, n is 2.

In a further possible subgroup of the compounds of formula I, Z is a single bond.

In a further possible subgroup of the compounds of formula I, R₂ is H.

In a further possible subgroup of the compounds of formula I, R₃ is H.

In a further possible subgroup of the compounds of formula I,
R₄ is, independently at each occurrence, H, (C₁-C₆)alkyl, (C₂-C₆)alkenyl, hydroxy(C₂-C₆)alkenyl, (C₁-C₆)alkoxy(C₁-C₆)alkyl, (C₂-C₆)alkenyloxy(C₁-C₆)alkyl, (C₁-C₆)alkyl-(C═O)—, (C₁-C₆)alkoxy-(C═O)—, hydroxy(C₁-C₆)alkoxy(C₁-C₆)alkyl or (C₁-C₆)alkoxy(C₁-C₆)alkoxy(C₁-C₆)alkyl; for example, H.

In another possible subgroup of the compounds of formula I,
R₄ is, independently at each occurrence, H, hydroxy or (C₁-C₆)alkyl; for example, H.

In a further possible subgroup of the compounds of formula I, m is 0.

In another possible subgroup of the compounds of formula I, m is 1.

In a further possible subgroup of the compounds of formula I
R₁ is, independently at each occurrence, hydroxy, phenyl(C₁-C₆)alkoxy, (C₁-C₆)alkoxy-(C═O)—, CN, NO₂, NH₂, mono- or di(C₁-C₆)alkylamino or carboxy.

In a further possible subgroup of the compounds of formula I,
R₁ is, independently at each occurrence, hydroxy, halogen, phenyl(C₁-C₆)alkoxy or NO₂.

In another possible subgroup of the compounds of formula I,
R₁ is, independently at each occurrence, hydroxy, (C₁-C₆)alkyl, (C₁-C₆)alkoxy or halogen; for example, hydroxy or halogen.

In a further possible subgroup of the compounds of formula I,
X is C(R₅)(R₆).

In a further possible subgroup of the compounds of formula I,
R₅ is H.

In another possible subgroup of the compounds of formula I,
R₅ is hydroxy.

In another possible subgroup of the compounds of formula I,
R₄ and R₅ attached to adjacent carbon ring atoms form a bond between the carbon ring atoms to which they are attached.

In a further possible subgroup of the compounds of formula I,
R₆ is unsubstituted phenyl.

In another possible subgroup of the compounds of formula I,
R₆ is phenyl substituted with 1 or 2 substituent(s) R₉;
R₉ is, independently at each occurrence, hydroxy, (C₂-C₆)alkenyl, hydroxy(C₁-C₆)alkyl, halo(C₁-C₆)alkyl, hydroxy(C₂-C₆)alkenyl, hydroxy(C₁-C₆)alkoxy, halo(C₁-C₆)alkoxy, phenyl(C₁-C₆)alkoxy, (C₁-C₆)alkoxy(C₁-C₆)alkyl, (C₂-C₆)alkenyloxy(C₁-C₆)alkyl, (C₁-C₆)alkyl-(C═O)—, (C₁-C₆)alkoxy-(C═O)—, (C₁-C₆)alkoxy-(C═O)—(C₁-C₆)alkyl, (C₁-C₆)alkyl-S—, (C₁-C₆)alkyl-(C═O)—O—, (C₁-C₆)alkyl-(C═O)—O—(C₁-C₆)alkyl, hydroxy-(C═O)—(C₁-C₆)alkoxy, hydroxy-(C═O)—(C₁-C₆)alkoxy(C₁-C₆)alkyl, (C₁-C₆)alkoxy-(C═O)—(C₁-C₆)alkoxy(C₁-C₆)alkyl, (C₁-C₆)alkyl-S—(C₁-C₆)alkyl, hydroxy(C₁-C₆)alkyl-S—(C₁-C₆)alkyl, (C₁-C₆)alkoxy(C₁-C₆)alkyl-S—(C₁-C₆)alkyl, (C₁-C₆)alkyl-(C═O)—S—(C₁-C₆)alkyl, (C₁-C₆)alkoxy-(C═O)—(C₁-C₆)alkyl-S—(C₁-C₆)alkyl, (C₁-C₆)alkyl-S—(C₁-C₆)alkoxy(C₁-C₆)alkyl, hydroxy(C₁-C₆)alkyl-S—(C₁-C₆)alkoxy(C₁-C₆)alkyl, (C₁-C₆)alkoxy(C₁-C₆)alkyl-S—(C₁-C₆)alkoxy(C₁-C₆)alkyl, (C₁-C₆)alkyl-(O═S═O)—(C₁-C₆)alkyl, hydroxy(C₁-C₆)alkyl-(O═S═O)—(C₁-C₆)alkyl, (C₁-C₆)alkoxy(C₁-C₆)alkyl-(O═S═O)—(C₁-C₆)alkyl, (C₁-C₆)alkyl-(O═S═O)—(C₁-C₆)alkoxy(C₁-C₆)alkyl, hydroxy(C₁-C₆)alkyl-(O═S═O)—(C₁-C₆)alkoxy(C₁-C₆)alkyl, (C₁-C₆)alkoxy(C₁-C₆)alkyl-(O═S═O)(C₁-C₆)alkoxy(C₁-C₆)alkyl, (C₁-C₆)alkyl-O—(O═S═O)—(C₁-C₆)alkyl, hydroxy(C₁-C₆)alkyl-O—(O═S═O)—(C₁-C₆)alkyl, (C₁-C₆)alkyl-(O═S═O)—O—(C₁-C₆)alkyl, hydroxy(C₁-C₆)alkyl-(O═S═O)—O—(C₁-C₆)alkyl, (C₁-C₆)alkyl-(O═S═O)—N—(R₁₀)—(C₁-C₆)alkyl, hydroxy(C₁-C₆)alkoxy(C₁-C₆)alkyl, (C₁-C₆)alkoxy(C₁-C₆)alkoxy(C₁-C₆)alkyl, halo(C₁-C₆)alkoxy(C₁-C₆)alkyl, CN, NO₂, (R₁₀)₂N—, (R₁₀)₂N—(C₁-C₆)alkyl, (R₁₀)₂N—(C═O)—, (R₁₀)₂N—(C═O)—(C₁-C₆)alkoxy(C₁-C₆)alkyl, (R₁₀)₂N—(C═O)—(C₁-C₆)alky-S—(C₁-C₆)alkyl, (R₁₀)₂N—(C═O)—(C₁-C₆)alkyl-(O═S═O)—(C₁-C₆)alkyl, carboxy, R₁₁ (O═S═O)— or R₁₁—(O═S═O)—O—;
or
R₉ and R₉ attached to adjacent carbon ring atoms form, together with the carbon ring atoms to which they are attached, a condensed phenyl ring, a condensed 5 or 6 membered unsaturated carbocyclic ring or a condensed 5 or 6 membered unsaturated heterocyclic ring containing I or 2 heteroatom(s) selected from O and S, wherein said phenyl, carbocyclic or heterocyclic ring is unsubstituted or substituted with 1 or 2 substituent(s) each independently being hydroxy, (C₁-C₆)alkyl, (C₁-C₆)alkoxy, hydroxy(C₁-C₆)alkyl, halo(C₁-C₆)alkyl, (C₁-C₆)alkoxy(C₁-C₆)alkyl, (C₁-C₆)alkyl-(C═O)— or (C₁-C₆)alkoxy-(C═O)—.

In another possible subgroup of the compounds of formula I,
R₆ is phenyl substituted with 1 or 2 substituent(s) R₉;
R₉ is, independently at each occurrence, hydroxy, (C₁-C₆)alkyl, (C₁-C₆)alkoxy, halogen, hydroxy(C₁-C₆)alkyl, halo(C₁-C₆)alkyl, hydroxy(C₁-C₆)alkoxy, halo(C₁-C₆)alkoxy, phenyl(C₁-C₆)alkoxy, (C₁-C₆)alkoxy-(C═O)—, (C₁-C₆)alkyl-(C═O)—O—, hydroxy-(C═O)—(C₁-C₆)alkoxy, CN, (R₁₀)₂N—, (R₁₀)₂N—(C═O)—, R₁₁—(O═S═O)— or R₁₁—(O═S═O)—O—;
or
R₉ and R₉ attached to adjacent carbon ring atoms form, together with the carbon ring atoms to which they are attached, a condensed 5 membered unsaturated heterocyclic ring containing 2 heteroatoms selected from 0, wherein said heterocyclic ring is unsubstituted; for example, R₉ is phenyl substituted with 1 substituent R₉ and R₉ is hydroxy, (C₁-C₆)alkyl, (C₁-C₆)alkoxy, halogen, hydroxy(C₁-C₆)alkoxy or (R₁₀)₂N—.

In another possible subgroup of the compounds of formula I,
X is C(R₇)(R₈).

In a further possible subgroup of the compounds of formula I,
R₇ is (C₁-C₆)alkyl, hydroxy(C₁-C₆)alkyl, (C₁-C₆)alkoxy(C₁-C₆)alkyl or (C₁-C₆)alkoxy-(C═O)—; for example, (C₁-C₆)alkyl.

In a further possible subgroup of the compounds of formula I,
R₈ is (C₂-C₆)alkenyl, halogen, halo(C₁-C₆)alkyl, hydroxy(C₁-C₆)alkenyl, hydroxy(C₁-C₆)alkoxy, halo(C₁-C₆)alkoxy, phenyl(C₁-C₆)alkoxy, (C₁-C₆)alkoxy(C₁-C₆)alkyl, (C₂-C₆)alkenyloxy(C₁-C₆)alkyl, (C₁-C₆)alkyl-(C═O)—, (C₁-C₆)alkoxy-(C═O)—(C₁-C₆)alkyl, (C₁-C₆)alkyl-S—, hydroxy-(C═O)—(C₁-C₆)alkoxy, hydroxy-(C═O)—(C₁-C₆)alkoxy(C₁-C₆)alkyl, (C₁-C₆)alkoxy-(C═O)—(C₁-C₆)alkoxy(C₁-C₆)alkyl, (C₁-C₆)alkyl-S—(C₁-C₆)alkyl, hydroxy(C₁-C₆)alkyl-S—(C₁-C₆)alkyl, (C₁-C₆)alkoxy(C₁-C₆)alkyl-S—(C₁-C₆)alkyl, (C₁-C₆)alkyl-(C═O)—S—(C₁-C₆)alkyl, (C₁-C₆)alkoxy-(C═O)—(C)—C₆)alkyl-S—(C₁-C₆)alkyl, (C₁-C₆)alkyl-S—(C₁-C₆)alkoxy(C₁-C₆)alkyl, hydroxy(C₁-C₆)alkyl-S—(C₁-C₆)alkoxy(C₁-C₆)alkyl, (C₁-C₆)alkoxy(C₁-C₆)alkyl-S—(C₁-C₆)alkoxy(C₁-C₆)alkyl, (C₁-C₆)alkyl-(O═S═O)—(C₁-C₆)alkyl, hydroxy(C₁-C₆)alkyl-(O═S═O)—(C₁-C₆)alkyl, (C₁-C₆)alkoxy(C₁-C₆)alkyl-(O═S═O)—(C₁-C₆)alkyl, (C₁-C₆)alkyl-(O═S═O)—(C₁-C₆)alkoxy(C₁-C₆)alkyl, hydroxy(C₁-C₆)alkyl-(O═S═O)—(C₁-C₆)alkoxy(C₁-C₆)alkyl, (C₁-C₆)alkoxy(C₁-C₆)alkyl-(O═S═O)—(C₁-C₆)alkoxy(C₁-C₆)alkyl, (C₁-C₆)alkyl-O—(O═S═O)—(C₁-C₆)alkyl, hydroxy(C₁-C₆)alkyl-O—(O═S═O)—(C₁-C₆)alkyl, (C₁-C₆)alkyl-(O═S═O)—O—(C₁-C₆)alkyl, hydroxy(C₁-C₆)alkyl-(O═S═O)—O—(C₁-C₆)alkyl, (C₁-C₆)alkyl-(O═S═O)—N(R₁₀)—(C₁-C₆)alkyl, hydroxy(C₁-C₆)alkoxy(C₁-C₆)alkyl, (C₁-C₆)alkoxy(C₁-C₆)alkoxy(C₁-C₆)alkyl, halo(C₁-C₆)alkoxy(C₁-C₆)alkyl, CN, NO₂, (R₁₀)₂N—, (R₁₀)₂N—(C₁-C₆)alkyl, (R₁₀)₂N—(C═O)—(C₁-C₆)alkoxy(C₁-C₆)alkyl, (R₁₀)₂N—(C═O)—(C₁-C₆)alkyl-S—(C₁-C₆)alkyl, (R₁₀)₂N—(C═O)—(C₁-C₆)alkyl-(O═S═O)—(C₁-C₆)alkyl, R₁₁—(O═S═O)— or R₁₁—(O═S═O)—O—; for example, (C₂-C₆)alkenyl, halogen, hydroxy(C₂-C₆)alkenyl, hydroxy(C₁-C₆)alkoxy, halo(C₁-C₆)alkoxy, (C₂-C₆)alkenyloxy(C₁-C₆)alkyl, hydroxy-(C═O)—(C₁-C₆)alkoxy, hydroxy-(C═O)—(C₁-C₆)alkoxy(C₁-C₆)alkyl, (C₁-C₆)alkoxy-(C═O)—(C₁-C₆)alkoxy(C₁-C₆)alkyl, hydroxy(C₁-C₆)alkyl-S—(C₁-C₆)alkyl, (C₁-C₆)alkoxy(C₁-C₆)alkyl-S—(C₁-C₆)alkyl, (C₁-C₆)alkoxy-(C═O)—(C₁-C₆)alkyl-S—(C₁-C₆)alkyl, (C₁-C₆)alkyl-S—(C₁-C₆)alkoxy(C₁-C₆)alkyl, hydroxy(C₁-C₆)alkyl-S—(C₁-C₆)alkoxy(C₁-C₆)alkyl, (C₁-C₆)alkoxy(C₁-C₆)alkyl-S—(C₁-C₆)alkoxy(C₁-C₆)alkyl, hydroxy(C₁-C₆)alkyl-(O═S═O)—(C₁-C₆)alkyl, (C₁-C₆)alkoxy(C₁-C₆)alkyl-(O═S═O)—(C₁-C₆)alkyl, (C₁-C₆)alkyl-(O═S═O)—(C₁-C₆)alkoxy(C₁-C₆)alkyl, hydroxy(C₁-C₆)alkyl-(O═S═O)—(C₁-C₆)alkoxy(C₁-C₆)alkyl, (C₁-C₆)alkoxy(C₁-C₆)alkyl-(O═S═O)—(C₁-C₆)alkoxy(C₁-C₆)alkyl, (C₁-C₆)alkyl-O—(O═S═O)—(C₁-C₆)alkyl, hydroxy(C₁-C₆)alkyl-O—(O—S═O)—(C₁-C₆)alkyl, (C₁-C₆)alkyl-(O═S═O)—O—(C₁-C₆)alkyl, hydroxy(C₁-C₆)alkyl-(O═S═O)—O—(C₁-C₆)alkyl, (C₁-C₆)alkyl-(O═S═O)—N(R₁₀)—(C₁-C₆)alkyl, hydroxy(C₁-C₆)alkoxy(C₁-C₆)alkyl, (C₁-C₆)alkoxy(C₁-C₆)alkoxy(C₁-C₆)alkyl, CN, NO₂, (R₁₀)₂N—(C═O)—(C₁-C₆)alkoxy(C₁-C₆)alkyl, (R₁₀)₂N—(C═O)—(C₁-C₆)alkyl-S—(C₁-C₆)alkyl, (R₁₀)₂N—(C═O)—(C₁-C₆)alkyl-(O═S═O)—(C₁-C₆)alkyl or R₁₁—(O═S═O)—O—.

In another possible subgroup of the compounds of formula I,
R₈ is hydroxy, (C₁-C₆)alkyl, (C₂-C₆)alkenyl, halogen, hydroxy(C₁-C₆)alkyl, halo(C₁-C₆)alkyl, hydroxy(C₁-C₆)alkoxy, (C₁-C₆)alkoxy(C₁-C₆)alkyl, (C₂-C₆)alkenyloxy(C₁-C₆)alkyl, (C₁-C₆)alkyl-(C═O)—, (C₁-C₆)alkoxy-(C═O)—, (C₁-C₆)alkoxy-(C═O)—(C₁-C₆)alkyl, (C₁-C₆)alkyl-(C═O)—O—(C₁-C₆)alkyl, hydroxy-(C═O)—(C₁-C₆)alkoxy(C₁-C₆)alkyl, (C₁-C₆)alkoxy-(C═O)—(C₁-C₆)alkoxy(C₁-C₆)alkyl, hydroxy(C₁-C₆)alkyl-S—(C₁-C₆)alkyl, (C₁-C₆)alkyl-(C═O)—S—(C₁-C₆)alkyl, (C₁-C₆)alkyl-(O═S═O)—O—(C₁-C₆)alkyl, hydroxy(C₁-C₆)alkoxy(C₁-C₆)alkyl, (C₁-C₆)alkoxy(C₁-C₆)alkoxy(C₁-C₆)alkyl, halo(C₁-C₆)alkoxy(C₁-C₆)alkyl, CN, (R₁₀)₂N—(C₁-C₆)alkyl, (R₁₀)₂N—(C═O)— or carboxy; for example, hydroxy, (C₂-C₆)alkenyl, halogen, hydroxy(C₁-C₆)alkyl, halo(C₁-C₆)alkyl, hydroxy(C₁-C₆)alkoxy, (C₁-C₆)alkoxy(C₁-C₆)alkyl, (C₂-C₆)alkenyloxy(C₁-C₆)alkyl, (C₁-C₆)alkyl-(C═O)—, (C₁-C₆)alkoxy-(C═O)—(C₁-C₆)alkyl, (C₁-C₆)alkyl-(C═O)—O—(C₁-C₆)alkyl, hydroxy-(C═O)—(C₁-C₆)alkoxy(C₁-C₆)alkyl, (C₁-C₆)alkoxy-(C═O)—(C₁-C₆)alkoxy(C₁-C₆)alkyl, hydroxy(C₁-C₆)alkyl-S—(C₁-C₆)alkyl, (C₁-C₆)alkyl-(C═O)—S—(C₁-C₆)alkyl, (C₁-C₆)alkyl-(O═S═O)—O—(C₁-C₆)alkyl, hydroxy(C₁-C₆)alkoxy(C₁-C₆)alkyl, (C₁-C₆)alkoxy(C₁-C₆)alkoxy(C₁-C₆)alkyl, halo(C₁-C₆)alkoxy(C₁-C₆)alkyl, CN, (R₁₀)₂N—(C₁-C₆)alkyl, (R₁₀)₂N—(C═O)— or carboxy; such as hydroxy(C₁-C₆)alkyl, hydroxy(C₁-C₆)alkoxy, (C₁-C₆)alkoxy(C₁-C₆)alkyl or hydroxy(C₁-C₆)alkoxy(C₁-C₆)alkyl.

In another possible subgroup of the compounds of formula I,
R₄ and R₈ attached to adjacent carbon ring atoms form, together with the carbon ring atoms to which they are attached, a condensed phenyl ring, wherein said phenyl ring is unsubstituted or substituted with 1 or 2 substituent(s) each independently being hydroxy, (C₁-C₆)alkyl, (C₁-C₆)alkoxy, hydroxy(C₁-C₆)alkyl, halo(C₁-C₆)alkyl, (C₁-C₆)alkoxy(C₁-C₆)alkyl, (C₁-C₆)alkyl-(C═O)— or (C₁-C₆)alkoxy-(C═O)—; for example,
R₄ and R₈ attached to adjacent carbon ring atoms form, together with the carbon ring atoms to which they are attached, a condensed phenyl ring, wherein said phenyl ring is substituted with 1 substituent being hydroxy(C₁-C₆)alkyl or (C₁-C₆)alkoxy-(C═O)—.

In another possible subgroup of the compounds of formula I,
R₄ and R₈ attached to adjacent carbon ring atoms form, together with the carbon ring atoms to which they are attached, a condensed 5 or 6 membered saturated or unsaturated carbocyclic ring or a condensed 5 or 6 membered saturated or unsaturated heterocyclic ring containing 1 or 2 heteroatom(s) selected from N, O, and S, wherein said carbocyclic or heterocyclic ring is unsubstituted or substituted with I or 2 substituent(s) each independently being hydroxy, (C₁-C₆)alkyl, (C₁-C₆)alkoxy, hydroxy(C₁-C₆)alkyl, halo(C₁-C₆)alkyl, (C₁-C₆)alkoxy(C₁-C₆)alkyl, (C₁-C₆)alkyl-(C═O)—, (C₁-C₆)alkoxy-(C═O)— or oxo.

In yet another possible subgroup of the compounds of formula I,
R₄ and R₈ attached to adjacent carbon ring atoms form, together with the carbon ring atoms to which they are attached, a condensed 6 membered saturated carbocyclic ring, wherein said carbocyclic ring is unsubstituted.

In yet another possible subgroup of the compounds of formula I,
R₄ and R₈ attached to adjacent carbon ring atoms form, together with the carbon ring atoms to which they are attached, a condensed 5 or 6 membered saturated or unsaturated heterocyclic ring containing 1 or 2 heteroatom(s) selected from N, O, and S, wherein said heterocyclic ring is unsubstituted or substituted with 1 or 2 substituent(s) each independently being hydroxy, (C₁-C₆)alkyl, (C₁-C₆)alkoxy, hydroxy(C₁-C₆)alkyl, halo(C₁-C₆)alkyl, (C₁-C₆)alkoxy(C₁-C₆)alkyl, (C₁-C₆)alkyl-(C═O)—, (C₁-C₆)alkoxy-(C═O)— or oxo.

In another possible subgroup of the compounds of formula I,
R₇ and R₈ form, together with the carbon ring atom to which they are attached, a 5 or 6 membered saturated carbocyclic ring or a 5 or 6 membered saturated heterocyclic ring containing 1 or 2 heteroatom(s) selected from N, O, and S, wherein said carbocyclic or heterocyclic ring is unsubstituted or substituted with 1 or 2 substituent(s) each independently being hydroxy, (C₁-C₆)alkyl or oxo; for example,
R₇ and R₈ form, together with the carbon ring atom to which they are attached, a 5 or 6 membered saturated heterocyclic ring containing 1 or 2 heteroatom(s) selected from N, O, and S, wherein said heterocyclic ring is unsubstituted or substituted with 1 or 2 substituent(s) each independently being hydroxy, (C₁-C₆)alkyl or oxo.

In a further possible subgroup of the compounds of formula I,
X is C(R₅)(R₆) or C(R₇)(R₈);
Z is —[C(R₄)₂]n- or a single bond;
R₁ is, independently at each occurrence, hydroxy, (C₁-C₆)alkyl, (C₁-C₆)alkoxy, halogen, phenyl(C₁-C₆)alkoxy or NO₂;
R₂ is, independently at each occurrence, H;
R₃ is, independently at each occurrence, H;
R₄ is, independently at each occurrence, H, hydroxy or (C₁-C₆)alkyl;
or
R₄ and R₄ both attached to the same carbon ring atom form, together with the carbon ring atom to which they are attached, a —(C═O)— group;
R₅ is H;
or
R₄ and R₅ attached to adjacent carbon ring atoms form a bond between the carbon ring atoms to which they are attached;
R₆ is phenyl unsubstituted or substituted with 1 or 2 substituent(s) R₉;
R₇ is (C₁-C₆)alkyl, hydroxy(C₁-C₆)alkyl, (C₁-C₆)alkoxy(C₁-C₆)alkyl or (C₁-C₆)alkoxy-(C═O)—;
R₈ is hydroxy, (C₁-C₆)alkyl, (C₂-C₆)alkenyl, (C₁-C₆)alkoxy, halogen, hydroxy(C₁-C₆)alkyl, halo(C₁-C₆)alkyl, hydroxy(C₁-C₆)alkoxy, (C₁-C₆)alkoxy(C₁-C₆)alkyl, (C₂-C₆)alkenyloxy(C₁-C₆)alkyl, (C₁-C₆)alkyl-(C═O)—, (C₁-C₆)alkoxy-(C═O)—, (C₁-C₆)alkoxy-(C═O)—(C₁-C₆)alkyl, (C₁-C₆)alkyl-(C═O)—O—(C₁-C₆)alkyl, hydroxy-(C═O)—(C₁-C₆)alkoxy(C₁-C₆)alkyl, (C₁-C₆)alkoxy-(C═O)—(C₁-C₆)alkoxy(C₁-C₆)alkyl, hydroxy(C₁-C₆)alkyl-S—(C₁-C₆)alkyl, (C₁-C₆)alkyl-(C═O)—S—(C₁-C₆)alkyl, (C₁-C₆)alkyl-(O═S═O)—O—(C₁-C₆)alkyl, hydroxy(C₁-C₆)alkoxy(C₁-C₆)alkyl, (C₁-C₆)alkoxy(C₁-C₆)alkoxy(C₁-C₆)alkyl, halo(C₁-C₆)alkoxy(C₁-C₆)alkyl, CN, (R₁₀)₂N—(C₁-C₆)alkyl, (R₁₀)₂N—(C═O)—, carboxy, or (C₁-C₆)alkoxy-(C₁-C₆)alkoxy;
or
R₄ and R₈ attached to adjacent carbon ring atoms form, together with the carbon ring atoms to which they are attached, a condensed phenyl ring, wherein said phenyl ring is unsubstituted or substituted with 1 or 2 substituent(s) each independently being hydroxy(C₁-C₆)alkyl or (C₁-C₆)alkoxy-(C═O)—;
or R₄ and R₈ attached to adjacent carbon ring atoms form, together with the carbon ring atoms to which they are attached, a condensed 6 membered saturated carbocyclic ring, wherein said carbocyclic ring is unsubstituted;
R₉ is, independently at each occurrence, hydroxy, (C₁-C₆)alkyl, (_{C1}-C₆)alkoxy, halogen, hydroxy(C₁-C₆)alkyl, halo(C₁-C₆)alkyl, hydroxy(C₁-C₆)alkoxy, halo(C₁-C₆)alkoxy, phenyl(C₁-C₆)alkoxy, (C₁-C₆)alkoxy-(C═O)—, (C₁-C₆)alkyl-(C═O)—O—, hydroxy-(C═O)—(C₁-C₆)alkoxy, CN, (R₁₀)₂N—, (R₁₀)₂N—(C═O)—, R₁₁—(O═S═O)— or R₁₁—(O═S═O)—O—;
or
R₉ and R₉ attached to adjacent carbon ring atoms form, together with the carbon ring atoms to which they are attached, a condensed 5 or 6 membered unsaturated heterocyclic ring containing 1 or 2 heteroatom(s) selected from O, wherein said phenyl, carbocyclic or heterocyclic ring is unsubstituted;
R₁₀ is, independently at each occurrence, H or (C₁-C₆)alkyl,
R₁₁ is, independently at each occurrence, (C₁-C₆)alkyl or halo(C₁-C₆)alkyl;
m is 0, 1 or 2; and
n is 1 or 2.

In another possible subgroup of the compounds of formula I,
X is C(R₅)(R₆) or C(R₇)(R₈);
Z is —[C(R₄)₂]n-;
R₁ is, independently at each occurrence, hydroxy, (C₁-C₆)alkyl, or halogen;
R₂ is, independently at each occurrence, H;
R₃ is, independently at each occurrence, H;
R₄ is, independently at each occurrence, H;
R₅ is H;
R₆ is phenyl unsubstituted or substituted with 1 or 2 substituent(s) R₉;
R₇ is (C₁-C₆)alkyl;
R₈ is hydroxy(C₁-C₆)alkyl, hydroxy(C₁-C₆)alkoxy, (C₁-C₆)alkoxy(C₁-C₆)alkyl or hydroxy(C₁-C₆)alkoxy(C₁-C₆)alkyl,
R₉ is, independently at each occurrence, hydroxy, (C₁-C₆)alkyl, (C₁-C₆)alkoxy, halogen, hydroxy(C₁-C₆)alkoxy or (R₁₀)₂N—;
R₁₀ is, independently at each occurrence, H or (C₁-C₆)alkyl,
m is 0 or 1;
n is 1.

In yet another possible subgroup of the compounds of formula I,
X is C(R₅)(R₆) or C(R₇)(R₈);
Z is a single bond;
R₁ is, independently at each occurrence, hydroxy, (C₁-C₆)alkyl, or halogen;
R₂ is, independently at each occurrence, H;
R₃ is, independently at each occurrence, H;
R₄ is, independently at each occurrence, H;
R₅ is H;
R₆ is phenyl unsubstituted or substituted with 1 or 2 substituent(s) R₉;
R₇ is (C₁-C₆)alkyl;
R₈ is hydroxy(C₁-C₆)alkyl, hydroxy(C₁-C₆)alkoxy, (C₁-C₆)alkoxy(C₁-C₆)alkyl or hydroxy(C₁-C₆)alkoxy(C₁-C₆)alkyl,
R₉ is, independently at each occurrence, hydroxy, (C₁-C₆)alkyl, (C₁-C₆)alkoxy, halogen, hydroxy(C₁-C₆)alkoxy or (R₁₀)₂N—;
R₁₀ is, independently at each occurrence, H or (C₁-C₆)alkyl,
m is 0 or 1;
n is 1.

In a further possible subgroup of the compounds of formula I, the compound is 1-(2,3-dihydrobenzo[1,4]dioxin-2-ylmethyl)-5-phenyl-1,2,3,6-tetrahydropyridine, 1-(2,3-dihydrobenzo[1,4]dioxin-2-ylmethyl)-3-phenylpiperidine, 1-(2,3-dihydrobenzo[1,4]dioxin-2-ylmethyl)-3-(2-methoxyphenyl)piperidine, 3-(4-chlorophenyl)-1-(2,3-dihydrobenzo[1,4]dioxin-2-ylmethyl)piperidine, 1-(2,3-dihydrobenzo[1,4]dioxin-2-ylmethyl)-3-(3-methoxyphenyl)piperidine, 1-(2,3-dihydrobenzo[1,4]dioxin-2-ylmethyl)-3-p-tolylpiperidine, 1-(2,3-dihydrobenzo[1,4]dioxin-2-ylmethyl)-3-(4-methoxyphenyl)piperidine,1-(2,3-dihydrobenzo[1,4]dioxin-2-ylmethyl)-3-o-tolylpiperidine, 4-[1-(2,3-dihydrobenzo[1,4]dioxin-2-ylmethyl)piperidin-3-yl]phenol, 1-(2,3-dihydrobenzo[1,4]dioxin-2-ylmethyl)-3-(3-fluorophenyl)piperidine, 3-[1-(2,3-dihydrobenzo[1,4]dioxin-2-ylmethyl)piperidin-3-yl]phenol, 2-[1-(2,3-dihydrobenzo[1,4]dioxin-2-ylmethyl)piperidin-3-yl]phenol, (R*)-1-[(S)-1-(2,3-dihydrobenzo[1,4]dioxin-2-yl)methyl]-3-(2-fluorophenyl)piperidine, (S*)-1-[(S)-1-(2,3-dihydrobenzo[1,4]dioxin-2-yl)methyl]-3-(2-fluorophenyl)piperidine, (R*)-1-[(S)-1-(2,3-dihydrobenzo[1,4]dioxin-2-yl)methyl]-3-(3-fluorophenyl)piperidine, (R*)-1-[(S)-1-(2,3-dihydrobenzo[1,4]dioxin-2-yl)methyl]-3-(3-fluorophenyl)piperidine HCl, (S*)-1-[(S)-1-(2,3-dihydrobenzo[1,4]dioxin-2-yl)methyl]-3-(3-fluorophenyl)piperidine, (R*)-1-[(S)-1-(2,3-dihydrobenzo[1,4]dioxin-2-yl)methyl]-3-(4-fluorophenyl)piperidine, (S*)-1-[(S)-1-(2,3-dihydrobenzo[1,4]dioxin-2-yl)methyl]-3-(4-fluorophenyl)piperidine, (R*)-1-[(S)-1-(2,3-dihydrobenzo[1,4]dioxin-2-yl)methyl]-3-(3-trifluoromethyl-phenyl)piperidine, (S*)-1-[(S)-1-(2,3-dihydrobenzo[1,4]dioxin-2-yl)methyl]-3-(3-trifluoromethyl-phenyl)piperidine, (R*)-1-[(S)-1-(2,3-dihydrobenzo[1,4]dioxin-2-yl)methyl]-3-(4-trifluoromethylphenyl)piperidine, (S*)-1-[(S)-1-(2,3-dihydrobenzo[1,4]dioxin-2-yl)methyl]-3-(4-trifluoromethylphenyl)piperidine, (R*)-1-[(S)-1-(2,3-dihydrobenzo[1,4]dioxin-2-yl)methyl]-3-(3-methoxyphenyl)piperidine, (R*)-1-[(S)-1-(2,3-dihydrobenzo[1,4]dioxin-2-yl)methyl]-3-(3-methoxyphenyl)piperidine HCl, (S*)-1-[(S)-1-(2,3-dihydrobenzo[1,4]dioxin-2-yl)methyl]-3-(3-methoxyphenyl)piperidine, (R*)-3-{1-[(S)-1-(2,3-dihydrobenzo[1,4]dioxin-2-yl)methyl]piperidin-3-yl}phenol, (R*)-3-{1-[(S)-1-(2,3-dihydrobenzo[1,4]dioxin-2-yl)methyl]piperidin-3-yl}phenol HCl, (3-{(R*)-1-[(S)-1-(2,3-dihydrobenzo[1,4]dioxin-2-yl)methyl]piperidin-3-yl}phenyl)methanol, acetic acid 3-{(S)-1-[(R*)-1-(2,3-dihydrobenzo[1,4]dioxin-2-yl)methyl]piperidin-3-yl}phenyl ester HCl, 2-(3-{(R*)-1-[(S)-1-(2,3-dihydrobenzo[1,4]dioxin-2-yl)methyl]piperidin-3-yl}phenoxy)ethanol, 3-(3-{(R*)-1-[(S)-1-(2,3-dihydrobenzo[1,4]dioxin-2-yl)methyl]piperidin-3-yl}phenoxy)propan-1-ol, trifluoromethanesulfonic acid 3-{(R*)-1-[(S)-1-(2,3-dihydrobenzo[1,4]dioxin-2-yl)methyl]-piperidin-3-yl}phenyl ester, (3-{(R*)-1-[(S)-1-(2,3-dihydrobenzo[1,4]dioxin-2-yl)methyl]piperidin-3-yl}phenoxy)acetic acid, 3-{(R*)-1-[(S)-1-(2,3-dihydrobenzo[1,4]dioxin-2-yl)methyl]-piperidin-3-yl}-benzonitrile, 3-benzo[1,3]dioxol-5-yl-1-[(S)-1-(2,3-dihydrobenzo[1,4]dioxin-2-yl)methyl]-piperidine, 3-[(R*)-1-((S)-6-benzyloxy-2,3-dihydro-benzo[1,4]dioxin-2-ylmethyl)-piperidin-3-yl]-phenol, (S)-2-[(R*)-3-(3-hydroxy-phenyl)-piperidin-1-ylmethyl]-2,3-dihydro-benzo[1,4]dioxin-6-ol, 3-[(R*)-1-((S)-7-benzyloxy-2,3-dihydro-benzo[1,4]dioxin-2-ylmethyl)-piperidin-3-yl]-phenol, (S)-3-[(R*)-3-(3-hydroxyphenyl)-piperidin-1-ylmethyl]-2,3-dihydro-benzo[1,4]dioxin-6-ol, 3-[(R*)-1-((S)-8-benzyloxy-2,3-dihydrobenzo[1,4]dioxin-2-ylmethyl)piperidin-3-yl]phenol, (S)-3-[(R*)-3-(3-hydroxy-phenyl)-piperidin-1-ylmethyl]-2,3-dihydrobenzo[1,4]dioxin-5-ol, (R*)-3-[1-((S)-7-fluoro-2,3-dihydrobenzo[1,4]dioxin-2-ylmethyl)piperidin-3-yl]phenol, 1-(2,3-dihydrobenzo[1,4]dioxin-2-ylmethyl)-3-methylpiperidine-3-carboxylic acid ethyl ester, 1-(2,3-dihydrobenzo[1,4]dioxin-2-ylmethyl)-3-ethylpiperidine-3-carboxylic acid ethyl ester, [1-(2,3-dihydrobenzo[1,4]dioxin-2-ylmethyl)-3-methylpiperidin-3-yl]methanol, 1-(2,3-dihydrobenzo[1,4]dioxin-2-ylmethyl)-3-methoxymethyl-3-methylpiperidine, 1-(2,3-dihydrobenzo[1,4]dioxin-2-ylmethyl)-3-ethoxymethyl-3-methylpiperidine, 3-chloromethyl-1-(2,3-dihydrobenzo[1,4]dioxin-2-ylmethyl)-3-methylpiperidine, 2-[1-(2,3-dihydrobenzo[1,4]dioxin-2-ylmethyl)-3-methylpiperidin-3-yl]propan-2-ol, 1-(2,3-dihydrobenzo[1,4]dioxin-2-ylmethyl)-3-(1-methoxy-1-methylethyl)-3-methylpiperidine, 1-(2,3-dihydrobenzo[1,4]dioxin-2-ylmethyl)-3-hydroxymethyl-3-methylpiperidin-4-ol, acetic acid 1-(2,3dihydrobenzo[1,4]dioxin-2-ylmethyl)-3-methylpiperidin-3-ylmethyl ester, methanesulfonic acid 1-(2,3-dihydrobenzo[1,4]dioxin-2-ylmethyl)-3-methylpiperidin-3-ylmethyl ester, [1-(2,3-dihydrobenzo[1,4]dioxin-2-ylmethyl)-3-ethylpiperidin-3-yl]methanol, 1-(2,3-dihydrobenzo[1,4]dioxin-2-ylmethyl)-3-ethyl-3-methoxymethylpiperidine, 1-(2,3-dihydrobenzo[1,4]dioxin-2-ylmethyl)-3-methoxymethoxymethyl-3-methylpiperidine, 1-[1-(2,3-dihydrobenzo[1,4]dioxin-2-ylmethyl)-3-ethylpiperidin-3-yl]ethanone, 1-[1-(2,3-dihydrobenzo[1,4]dioxin-2-ylmethyl)-3-ethylpiperidin-3-yl]ethanol, 3-allyloxymethyl-1-(2,3-dihydrobenzo[1,4]dioxin-2-ylmethyl)-3-ethylpiperidine, 2-[1-(2,3-dihydrobenzo[1,4]dioxin-2-ylmethyl)-3-ethylpiperidin-3-yl-methoxy]ethanol, 3-allyloxymethyl-1-(2,3-dihydrobenzo[1,4]dioxin-2-ylmethyl)-3-methyl-piperidine, 3-allyl-1-(2,3-dihydrobenzo[1,4]dioxin-2-ylmethyl)-piperidine-3-carboxylic acid ethyl ester, [3-allyl-1-(2,3-dihydrobenzo[1,4]dioxin-2-ylmethyl)piperidin-3-yl]methanol, 3-allyl-1-(2,3-dihydrobenzo[1,4]dioxin-2-ylmethyl)-3-methoxymethylpiperidine, 3-allyl-1-(2,3-dihydrobenzo[1,4]dioxin-2-ylmethyl)-3-ethoxymethylpiperidine, 1-(2,3-dihydrobenzo[1,4]dioxin-2-ylmethyl)-3-methyl-3-(2,2,2-trifluoroethoxymethyl)-piperidine, 2-[1-(2,3-dihydrobenzo[1,4]dioxin-2-ylmethyl)-3-methylpiperidin-3-ylmethoxy]ethanol, (S)-1-((R)-2,3-dihydrobenzo[1,4]dioxine-2-carbonyl)-3-methylpiperidine-3-carboxylic acid ethyl ester, lithium (S)-1-[(S)-1-(2,3-dihydrobenzo[1,4]dioxin-2-yl)methyl]-3-methyl-piperidine-3-carboxylate, {(S)-1-[(S)-1-(2,3-dihydrobenzo[1,4]dioxin-2-yl)methyl]-3-methylpiperidin-3-yl}methanol, 2-{(S)-1-[(S)-1-(2,3-dihydrobenzo[1,4]dioxin-2-yl)methyl]-3-methylpiperidin-3-ylmethoxy}-ethanol, 2-{(S)-1-[(S)-1-(2,3-dihydrobenzo[1,4]dioxin-2-yl)methyl]-3-methylpiperidin-3-ylmethoxy}-ethanol D-tartrate, (S)-1-[(S)-1-(2,3-dihydrobenzo[1,4]dioxin-2-yl)methyl]-3-(2-methoxyethoxymethyl)-3-methylpiperidine, (S)-1-[(S)-1-(2,3-dihydrobenzo[1,4]dioxin-2-yl)methyl]-3-methyl-3-(2,2,2-trifluoroethoxymethyl)piperidine, methanesulfonic acid (S)-1-[(S)-1-(2,3-dihydrobenzo[1,4]dioxin-2-yl)methyl]-3-methyl-piperidin-3-ylmethyl ester, thioacetic acid S—{(S)-1-[(S)-1-(2,3-dihydrobenzo[1,4]dioxin-2-yl)methyl]-3-methylpiperidin-3-ylmethyl}ester, 2-{(S)-1-[(S)-1-(2,3-dihydrobenzo[1,4]dioxin-2-yl)methyl]-3-methylpiperidin-3-ylmethyl-sulfanyl}ethanol, {(S)-1-[(S)-1-(2,3-dihydrobenzo[1,4]dioxin-2-yl)methyl]-3-methylpiperidin-3-ylmethoxy}acetic acid tert-butyl ester, sodium {(S)-1-[(S)-1-(2,3-dihydrobenzo[1,4]dioxin-2-yl)methyl]-3-methylpiperidin-3-yl-methoxy}acetate, 2-[(S)-1-((S)-7-fluoro-2,3-dihydrobenzo[1,4]dioxin-2-ylmethyl)-3-methylpiperidin-3-yl-methoxy]ethanol, 1-(2,3-dihydrobenzo[1,4]dioxin-2-ylmethyl)-3-fluoro-piperidine-3-carboxylic acid ethyl ester,[1-(2,3-dihydrobenzo[1,4]dioxin-2-ylmethyl)-3-fluoro-piperidin-3-yl]methanol, (S)-1-[(S)-1-(2,3-dihydro-benzo[1,4]dioxin-2-yl)methyl]-3-methyl-piperidine-3-carboxylic acid ethyl ester, [(S)-1-((S)-7-fluoro-2,3-dihydro-benzo[1,4]dioxin-2-ylmethyl)-3-methyl-piperidin-3-yl]-methanol, (S)-1-((S)-7-fluoro-2,3-dihydro-benzo[1,4]dioxin-2-ylmethyl)-3-methoxymethyl-3-methyl-piperidine, (S)-1-((S)-7-fluoro-2,3-dihydro-benzo[1,4]dioxin-2-ylmethyl)-3-methoxymethyl-3-methyl-piperidine-HCl, (S)-1-[(S)-1-(2,3-dihydro-benzo[1,4]-dioxin-2-yl)methyl]-3-methoxymethyl-3-methyl-piperidine, 3-{(R*)-1-[(S)-1-(2,3-dihydro-benzo[1,4]dioxin-2-yl)methyl]-piperidin-3-yl}-phenylamine, (R*)-3-{1-[(S)-1-(2,3-dihydrobenzo[1,4]dioxin-2-yl)methyl]piperidin-3-yl}phenol oxalate, (S)-2-[(R*)-3-(3-hydroxy-phenyl)-piperidin-1-ylmethyl]-2,3-dihydro-benzo[1,4]dioxin-5-ol, 1-[(S)-1-(2,3-dihydro-benzo[1,4]dioxin-2-yl)methyl]-3-(2-methoxy-phenyl)-pyrrolidine, (S)-1-[(S)-1-(2,3-dihydro-benzo[1,4]dioxin-2-yl)methyl]-3-(2-fluoro-ethoxymethyl)-3-methyl-piperidine HCl, (R*)-1-[(S)-1-(2,3-dihydro-benzo[1,4]dioxin-2-yl)methyl]-3-(3-fluoromethoxy-phenyl)-piperidine, 1-(2,3-dihydro-benzo[1,4]dioxin-2-ylmethyl)-3-methyl-pyrrolidine-3-carboxylic acid methyl ester, [1-(2,3-dihydro-benzo[1,4]dioxin-2-ylmethyl)-3-methylpyrrolidin-3-yl]-methanol, 2-[1-(2,3-dihydro-benzo[1,4]dioxin-2-ylmethyl)-3-methyl-pyrrolidin-3-ylmethoxy]-ethanol, 1-(2,3-dihydro-benzo[1,4]dioxin-2-ylmethyl)-3-methoxymethyl-3-methyl-pyrrolidine or 3-[(R)-1-((S)-7-nitro-2,3-dihydrobenzo[1,4]dioxin-2-ylmethyl)-piperidin-3-yl]-phenol.

It is evident to a person skilled in the art that, in the compounds of formula I, when the substituents R₉ and R₉ attached to adjacent carbon ring atoms form, together with the carbon ring atoms to which they are attached, a condensed 5 or 6 membered unsaturated carbocyclic ring or a condensed 5 or 6 membered unsaturated heterocyclic ring, said carbocyclic ring or heterocyclic ring may have further unsaturated bonds in addition to the unsaturated bond between the carbon ring atoms, to which said substituents are attached.

The terms employed herein have the meanings indicated below. The term “at least one” employed in the meanings below refers to one or several, such as one. For example, the term “at least one halogen” refers to one or several halogens, such as one halogen.

The term “hydroxy”, as employed herein as such or as part of another group, refers to a —OH group.

The term “(C₁-C₆)alkyl”, as employed herein as such or as part of another group, refers to a straight or branched chain saturated hydrocarbon group having 1, 2, 3, 4, 5 or 6 carbon atom(s). Representative examples of (C₁-C₆)alkyl include, but are not limited to, methyl, ethyl, n-propyl, iso-propyl, n-butyl, iso-butyl, sec-butyl, tert-butyl, n-pentyl, iso-pentyl, and n-hexyl.

The term “(C₁-C₆)alkoxy”, as employed herein as such or as part of another group, refers to an (C₁-C₆)alkyl group, as defined herein, appended to the parent molecular moiety through an oxygen atom. Representative examples of (C₁-C₆)alkoxy include, but are not limited to, methoxy, ethoxy, n-propoxy, n-butoxy, iso-butoxy, sec-butoxy, tert-butoxy, 2,2-dimethylpropoxy, 3-methylbutoxy, and n-hexoxy.

The term “halo” or “halogen”, as employed herein as such or as part of another group, refers to fluorine, chlorine, bromine or iodine.

The term “halo(C₁-C₆)alkyl”, as employed herein, refers to at least one halogen, as defined herein, appended to the parent molecular moiety through an (C₁-C₆)alkyl group, as defined herein. When there are several halogens, the halogens can be identical or different. Representative examples of halo(C₁-C₆)alkyl include, but are not limited to, fluoromethyl, chloromethyl, difluoromethyl, trifluoromethyl, 2-chloroethyl, 3-bromopropyl, and 2-chloropropyl.

The term “phenyl(C₁-C₆)alkoxy”, as employed herein, refers to at least one phenyl group appended to the parent molecular moiety through an (C₁-C₆)alkoxy group, as defined herein. Representative examples of phenyl(C₁-C₆)alkoxy include, but are not limited to, phenylmethoxy, 2-phenylethoxy, and 3-phenylpropoxy.

The term “amino”, as employed herein as part of another group, refers to a —NH₂ group.

The term “mono(C₁-C₆)alkylamino”, as employed herein, refers to one (C₁-C₆)alkyl group, as defined herein, appended to the parent molecular moiety through an amino group, as defined herein. Representative examples of mono(C₁-C₆)alkylamino include, but are not limited to, N-methylamino, N-ethylamino, and N-butylamino.

The term “di(C₁-C₆)alkylamino”, as employed herein, refers to two (C₁-C₆)alkyl groups, as defined herein, appended to the parent molecular moiety through an amino group, as defined herein. The (C₁-C₆)alkyl groups can be identical or different. Representative examples of di(C₁-C₆)alkylamino include, but are not limited to, N,N-dimethylamino and N,N-diethylamino.

The term “carboxy”, as employed herein, refers to a —COOH group.

The term “(C₂-C₆)alkenyl”, as employed herein as such or as part of another group, refers to a straight or branched chain hydrocarbon group having 2, 3, 4, 5 or 6 carbon atoms and containing at least one carbon-carbon double bond. Representative examples of (C₂-C₆)alkenyl include, but are not limited to, ethenyl and prop-2-enyl.

The term “hydroxy(C₁-C₆)alkyl”, as employed herein as such or as part of another group, refers to at least one hydroxy group, as defined herein, appended to the parent molecular moiety through an (C₁-C₆)alkyl group, as defined herein. Representative examples of hydroxy(C₁-C₆)alkyl include, but are not limited to, hydroxymethyl, 1-hydroxyethyl, 2-hydroxyethyl, 2,2-dihydroxyethyl, 1-hydroxypropyl, 3-hydroxypropyl, 1-hydroxy-1-methylethyl, and 1-hydroxy-1-methylpropyl.

The term “hydroxy(C₂-C₆)alkenyl”, as employed herein, refers to at least one hydroxy group, as defined herein, appended to the parent molecular moiety through an (C₂-C₆)alkenyl group, as defined herein. Representative examples of hydroxy(C₂-C₆)alkenyl include, but are not limited to, 1-hydroxyethenyl, 2-hydroxyethenyl, and 1-hydroxyprop-2-enyl.

The term “(C₁-C₆)alkoxy(C₁-C₆)alkyl”, as employed herein as such or as part of another group, refers to at least one (C₁-C₆)alkoxy group, as defined herein, appended to the parent molecular moiety through an (C₁-C₆)alkyl group, as defined herein. When there are several (C₁-C₆)alkoxy groups, the (C₁-C₆)alkoxy groups can be identical or different. Representative examples of (C₁-C₆)alkoxy(C₁-C₆)alkyl include, but are not limited to, methoxymethyl, ethoxymethyl, propoxymethyl, 2-methoxyethyl, 2-ethoxyethyl, 2,2-dimethoxyethyl, 1-methyl-2-propoxyethyl, 1-methoxy-1-methylethyl, and 4-methoxybutyl.

The term “(C₂-C₆)alkenyloxy”, as employed herein as part of another group, refers to an (C₂-C₆)alkenyl group, as defined herein, appended to the parent molecular moiety through an oxygen atom. Representative examples of (C₂-C₆)alkenyloxy include, but are not limited to, ethenyloxy, prop-2-enyloxy, bute-2-nyloxy, and hex-3-enyloxy.

The term “(C₂-C₆)alkenyloxy(C₁-C₆)alkyl”, as employed herein, refers to at least one (C₂-C₆)alkenyloxy group, as defined herein, appended to the parent molecular moiety through an (C₁-C₆)alkyl group, as defined herein. When there are several (C₂-C₆)alkenyloxy groups, the (C₂-C₆)alkenyloxy groups can be identical or different. Representative examples of (C₂-C₆)alkenyloxy(C₁-C₆)alkyl include, but are not limited to, prop-2-enyloxymethyl and ethenyloxyethyl.

The term “hydroxy(C₁-C₆)alkoxy”, as employed herein as such or as part of another group, refers to at least one hydroxy group, as defined herein, appended to the parent molecular moiety through an (C₁-C₆)alkoxy group, as defined herein. Representative examples of hydroxy(C₁-C₆)alkoxy include, but are not limited to, hydroxymethoxy, dihydroxymethoxy, 2-hydroxyethoxy, 2-hydroxypropoxy, 3-hydroxypropoxy, 2-hydroxybutoxy, and 2-hydroxy-1-methylethoxy.

The term “(C₁-C₆)alkoxy(C₁-C₆)alkoxy”, as employed herein as such or as part of another group, refers to at least one (C₁-C₆)alkoxy group, as defined herein, appended to the parent molecular moiety through an (C₁-C₆)alkoxy group, as defined herein. The (C₁-C₆)alkoxy groups can be identical or different. Representative examples of (C₁-C₆)alkoxy(C₁-C₆)alkoxy include, but are not limited to, methoxymethoxy, propoxymethoxy, 2-methoxyethoxy, 2-ethoxyethoxy, 2-butoxyethoxy, 2,2-dimethoxyethoxy, 1-methyl-2-propoxyethoxy, 2-methoxypropoxy and 4-methoxybutoxy.

The term “halo(C₁-C₆)alkoxy”, as employed herein as such or as part of another group, refers to at least one halogen, as defined herein, appended to the parent molecular moiety through an (C₁-C₆)alkoxy group, as defined herein. When there are several halogens, the halogens can be identical or different. Representative examples of halo(C₁-C₆)alkoxy include, but are not limited to, fluoromethoxy, chloromethoxy, difluoromethoxy, trifluoromethoxy, 2-bromoethoxy, 2,2,2-trichloroethoxy, 3-bromopropoxy, 2-chloropropoxy, and 4-chlorobutoxy.

The term “oxo”, as employed herein, refers to a ═O group.

The expression “compounds of the invention” as employed herein refers to the compounds of formula I.

Compounds of the invention are the compounds of the formula (I) and the salts, solvates and solvates of the salts thereof, the compounds that are encompassed by formula (I) and are of the formulae mentioned below and the salts, solvates and solvates of the salts thereof and the compounds that are encompassed by formula (I) and are cited below as working examples and the salts, solvates and solvates of the salts thereof if the compounds that are encompassed by formula (I) and are mentioned below are not already salts, solvates and solvates of the salts.

Compounds of the invention are likewise N-oxides and S-oxides of the compounds of the formula (I) and the salts, solvates and solvates of the salts thereof.

Preferred salts in the context of the present invention are physiologically acceptable salts of the compounds according to the invention. Also encompassed are salts which are not themselves suitable for pharmaceutical applications but can be used, for example, for the isolation, purification or storage of the compounds of the invention.

A suitable pharmaceutically acceptable salt of the compounds of the present invention may be, for example, an acid-addition salt of a compound of the present invention bearing a sufficiently basic nitrogen atom in a chain or in a ring, such as an acid-addition salt with an inorganic acid, or “mineral acid”, such as hydrochloric acid, hydrobromic acid, hydroiodic acid, sulfuric acid, sulfamic acid, disulfuric acid, phosphoric acid or nitric acid, for example, or with an organic acid such as formic acid, acetic acid, acetoacetic acid, pyruvic acid, trifluoroacetic acid, propionic acid, butyric acid, hexanoic acid, heptanoic acid, undecanoic acid, lauric acid, benzoic acid, salicylic acid, 2-(4-hydroxybenzoyl)benzoic acid, camphoric acid, cinnamic acid, cyclopentanepropionic acid, digluconic acid, 3-hydroxy-2-naphthoic acid, nicotinic acid, pamoic acid, pectinic acid, 3-phenylpropionic acid, pivalic acid, 2-hydroxyethanesulfonic acid, itaconic acid, trifluoromethanesulfonic acid, dodecylsulfuric acid, ethanesulfonic acid, benzenesulfonic acid, para-toluenesulfonic acid, methanesulfonic acid, 2-naphthalenesulfonic acid, naphthalenedisulfonic acid, camphorsulfonic acid, citric acid, tartaric acid, stearic acid, lactic acid, oxalic acid, malonic acid, succinic acid, malic acid, adipic acid, alginic acid, maleic acid, fumaric acid, D-gluconic acid, mandelic acid, ascorbic acid, glucoheptanoic acid, glycerophosphoric acid, aspartic acid, sulfosalicylic acid or thiocyanic acid, for example.

Further, another suitable pharmaceutically acceptable salt of a sufficiently acidic compound of the present invention is an alkali metal salt, for example a sodium or potassium salt, an alkaline earth metal salt, for example a calcium, magnesium or strontium salt, or an aluminum or zinc salt, or an ammonium salt derived from ammonia or from an organic primary, secondary or tertiary amine having 1 to 20 carbon atoms, such as ethylamine, diethylamine, triethylamine, ethyldiisopropylamine, monoethanolamine, diethanolamine, triethanolamine, dicyclohexylamine, dimethylaminoethanol, diethylaminoethanol, tris(hydroxymethyl)aminomethane, procaine, dibenzylamine, N-methylmorpholine, arginine, lysine, 1,2-ethylenediamine, N-methylpiperidine, N-methylglucamine, N,N-dimethylglucamine, N-ethylglucamine, 1,6-hexanediamine, glucosamine, sarcosine, serinol, 2-amino-1,3-propanediol, 3-amino-1,2-propanediol, 4-amino-1,2,3-butanetriol, or a salt with a quarternary ammonium ion having 1 to 20 carbon atoms, such as tetramethylammonium, tetraethylammonium, tetra(n-propyl)ammonium, tetra(n-butyl)ammonium, N-benzyl-N,N,N-trimethylammonium, choline or benzalkonium.

Those skilled in the art will further recognize that it is possible for acid addition salts of the claimed compounds to be prepared by reaction of the compounds with the appropriate inorganic or organic acid via any of a number of known methods. Alternatively, alkali and alkaline earth metal salts of acidic compounds of the present invention are prepared by reacting the compounds of the present invention with the appropriate base via a variety of known methods.

The present invention includes all possible salts of the compounds of the present invention as single salts, or as any mixture of said salts, in any ratio.

Pharmaceutically acceptable esters, when applicable, may be prepared by known methods using pharmaceutically acceptable acids that are conventional in the field of pharmaceuticals and that retain the pharmacological properties of the free form. Nonlimiting examples of these esters include esters of aliphatic or aromatic alcohols. Representative examples of pharmaceutically acceptable esters include, but are not limited to, methyl, ethyl, n-propyl, iso-propyl, n-butyl, iso-butyl, sec-butyl, tert-butyl, and benzyl esters.

The invention includes within its scope all the possible geometric isomers, e.g. Z and E isomers (cis and transisomers), of the compounds as well as all the possible optical isomers, e.g. diastereomers and enantiomers, of the compounds. Furthermore, the invention includes in its scope both the individual isomers and any mixtures thereof, e.g. racemic mixtures. The individual isomers may be obtained using the corresponding isomeric forms of the starting material or they may be separated after the preparation of the end compound according to conventional separation methods.

For the separation of optical isomers, e.g. enantiomers, from the mixture thereof, conventional resolution methods, e.g. fractional crystallization, may be used.

The compounds of formula (I) and (II), their production and their action as alpha2C antagonists for the treatment of diseases or conditions of the peripheric or central nervous system are disclosed in WO2009013390 in general and especially the compounds specifically are an explicit part of the description of the present invention and are hereby incorporated by reference.

The term effective amount as used herein refers to an amount of a compound of formula (I) that is effective for treatment and/or prophylaxis of sleep-related breathing disorders, preferably obstructive and central sleep apneas and snoring.

The present invention relates to (alpha-2C) antagonists, in particular 3-substituted 1-(2,3-dihydrobenzo[1,4]dioxin-2-ylmethyl)azacycles of formula (I) for the use in a method for the treatment and/or prophylaxis of sleep-related breathing disorders, preferably obstructive and central sleep apneas and snoring.

The present invention further relates to the use of compounds of formula (I) for the manufacture of medicaments for the treatment and/or prophylaxis of sleep-related breathing disorders, preferably obstructive and central sleep apneas and snoring.

A further subject of the present invention is the use of a combination of one or more compounds of the formula (I) with one or more other active compounds in a method for the treatment and/or prophylaxis sleep-related breathing disorders, preferably obstructive and central sleep apneas and snoring.

A further subject of the present invention is a pharmaceutical composition comprising at least one compounds of the formula (I) in combination with one or more inert non-toxic pharmaceutically suitable excipients for use in a method for the treatment and/or prophylaxis sleep-related breathing disorders, preferably obstructive and central sleep apneas and snoring.

The present invention further relates to pharmaceutical composition comprising a combination with one or more other active compounds in combination with one or more inert non-toxic pharmaceutically suitable excipients for use in a method for the treatment and/or prophylaxis sleep-related breathing disorders, preferably obstructive and central sleep apneas and snoring.

The present invention is also directed to a method for the treatment and/or prophylaxis of sleep-related breathing disorders, by administering systemically and/or locally a therapeutically effective amount of at least one compound of formula (I) or a medicament comprising at least one compound of formula (I) in combination with a inert, non-toxic, pharmaceutically acceptable additive.

A further subject of the present invention is a combination of one or more compounds of the formula (I) with one or more other active compounds for use in a method for the treatment and/or prophylaxis sleep-related breathing disorders, preferably obstructive and central sleep apneas and snoring. 3-substituted 1-(2,3-dihydrobenzo[1,4]dioxin-2-ylmethyl)azacycles of formula (I) according to the invention can be used alone or, if required, in combination with one or more other pharmacologically active substances, provided that this combination does not lead to undesirable and unacceptable side effects. Preferred examples of combination suitable for the purpose to treat sleep-related breathing disorders, preferably obstructive and central sleep apneas and snoring, include:
respiratory stimulants such as, by way of example and with preference, theophylline, doxapram, nikethamide or caffeine;
psychostimulants such as, by way of example and with preference, modafinil or armodafinil;
amphetamines and amphetamine derivatives such as, by way of example and with preference, amphetamine, metamphetamine or methylphenidate;
serotonin reuptake inhibitors such as, by way of example and with preference, fluoxetine, paroxetine, citalopram, escitalopram, sertraline, fluvoxamine or trazodone;
serotonin precursors such as, by way of example and with preference, L-tryptophan;
selective serotonin noradrenaline reuptake inhibitors such as, by way of example and with preference, venlafaxine or duloxetine;
noradrenergic and specific serotonergic antidepressants such as, by way of example and with preference, mirtazapine;
selective noradrenaline reuptake inhibitors such as, by way of example and with preference, reboxetine or atomoxetine;
tricyclic antidepressants such as, by way of example and with preference, amitriptyline, protriptyline, doxepine, trimipramine, imipramine, clomipramine or desipramine;
muscarinic receptor antagonists, by way of example and with preference oxybutynin;
GABA agonists such as, by way of example and with preference, baclofen;
glucocorticoids such as, by way of example and with preference, fluticasone, budesonide, beclometasone, mometasone, tixocortol or triamcinolone;
cannabinoid receptor agonists;
TASK-channel blockers by way of example and with preference TASK-channel blockers disclosed in WO 2017/097792 A1, WO 2017/097671 A1, WO 2018/015196 A1, WO 2018/228907 A1 and WO 2018/228909 A1 in general and especially;
carboanhydrase inhibitors such as, by way of example and with preference, acetazolamide, methazolamide or diclofenamide;
opioid and benzodiazepine receptor antagonists such as, by way of example and with preference, flumazenil, naloxone or naltrexone;
cholinesterase inhibitors such as, by way of example and with preference, neostigmine, pyridostigmine, physostigmine donepezil, galantamine or by way of example and with preference rivastigmine;
appetite suppressants such as, by way of example and with preference, sibutramine, topiramate, phentermine, lipase inhibitors or cannabinoid receptor antagonists;
mineralocorticoid receptor antagonists.

A preferred subject of the present invention is a combination of one or more compounds of the formula (I) or (II) with one or more other active compounds selected from the groups consisting of muscarinic receptor antagonists, mineralocorticoid receptor antagonists, TASK-channel blockers, diuretics, corticosteroids for use in a method for the treatment and/or prophylaxis sleep-related breathing disorders, preferably obstructive and central sleep apneas and snoring.

In a preferred embodiment of the invention, the compounds of the invention are administered in combination with a muscarinic receptor antagonist, by way of example and with preference oxybutynin.

In a preferred embodiment of the invention, the compounds of the invention are administered in combination with a TASK-channel blocker, by way of example and with preference WO 2017/097792 A1, WO 2017/097671 A1, WO 2018/015196 A1, WO 2018/228907 A1 and WO 2018/228909 A1 in general and especially.

In a preferred embodiment of the invention, the compounds of the invention are administered in combination with a mineralocorticoid receptor antagonist, by way of example and with preference spironolactone, eplerenone or finerenone.

In a preferred embodiment of the invention, the compounds of the invention are administered in combination with a diuretic, by way of example and with preference furosemide, bumetanide, torsemide, bendroflumethiazide, chlorothiazide, hydrochlorothiazide, hydroflumethiazide, methylclothiazide, polythiazide, trichloromethiazide, chlorthalidone, indapamide, metolazone, quinethazone, acetazolamide, dichlorphenamide, methazolamide, glycerol, isosorbide, mannitol, amiloride or triamterene.

In a preferred embodiment of the invention, the compounds of the invention are administered in combination with a corticosteroid, by way of example and with preference prednisone, prednisolone, methylprednisolone, triamcinolone, dexamethasone, betamethasone, beclomethasone, flunisolide, budesonide or fluticasone.

If required, 3-substituted 1-(2,3-dihydrobenzo[1,4]dioxin-2-ylmethyl)azacycles of formula (I) according to the invention can also be employed in conjunction with the use of one or more medical technical devices or auxiliaries, provided this does not lead to unwanted and unacceptable side-effects. Medical devices and auxiliaries suitable for such a combined application are, by way of example and with preference:
devices for positive airway pressure ventilation such as, by way of example and with preference, CPAP (continuous positive airway pressure) devices, BiPAP (bilevel positive airway pressure) devices and IPPV (intermittent positive pressure ventilation) devices;
neurostimulators of the Nervus hypoglossus;
intraoral auxiliaries such as, by way of example and with preference, protrusion braces;
nasal disposable valves;
nasal stents.

3-substituted 1-(2,3-dihydrobenzo[1,4]dioxin-2-ylmethyl)azacycles of formula (I) according to the invention can act systemically and/or locally. For this purpose, they can be administered in a suitable manner, for example by the oral, parenteral, pulmonal, intrapulmonal (inhalative), nasal, intranasal, pharyngeal, lingual, sublingual, buccal, rectal, dermal, transdermal, conjunctival or otic route, or as an implant or stent.

A further subject of the present invention is a pharmaceutical composition comprising a compound of the formula (I) for the systemically and/or locally administration by the oral, parenteral, pulmonal, intrapulmonal (inhalative), nasal, intranasal, pharyngeal, lingual, sublingual, buccal, rectal, dermal, transdermal, conjunctival or otic route, or as an implant or stent. The preferred administration is the oral route.

For these administration routes, the compounds according to the invention can be administered in suitable administration forms.

For oral administration, administration forms which function according to the state of the art, releasing the compounds according to the invention rapidly and/or in a modified manner, which contain the compounds according to the invention in crystalline and/or amorphized and/or dissolved form, such as for example tablets (uncoated or coated tablets, for example with gastric juice-resistant or delayed dissolution or insoluble coatings, which control the release of the compound according to the invention), tablets rapidly disintegrating in the oral cavity or films/wafers, films/lyophilisates, capsules (for example hard or soft gelatine capsules), dragees, granules, pellets, powders, emulsions, suspensions, aerosols or solutions are suitable.

Parenteral administration can be effected omitting an absorption step (e.g. intravenous, intra-arterial, intracardial, intraspinal or intralumbar administration) or involving absorption (e.g. intra-muscular, subcutaneous, intracutaneous, percutaneous or intraperitoneal administration). Suitable administration forms for parenteral administration include injection and infusion preparations in the form of solutions, suspensions, emulsions, lyophilisates or sterile powders.

For the other administration routes, for example inhalation formulations (including powder inhalers and nebulisers), nasal drops, solutions or sprays, tablets for lingual, sublingual or buccal administration, tablets, films/wafers or capsules, suppositories, oral or ophthalmic preparations, vaginal capsules, aqueous suspensions (lotions, shakable mixtures), lipophilic suspensions, ointments, creams, transdermal therapeutic systems (e.g. plasters), milk, pastes, foams, dusting powders, implants or stents are suitable.

Oral or parenteral administration, in particular oral and intravenous administration, are preferred.

The compounds according to the invention can be converted into the stated administration forms. This can be effected in a manner known per se by mixing with inert, non-toxic, pharmaceutically suitable additives. These additives include carriers (for example microcrystalline cellulose, lactose, mannitol), solvents (e.g. liquid polyethylene glycols), emulsifiers and dispersants or wetting agents (for example sodium dodecylsulphate, polyoxysorbitan oleate), binders (for example polyvinylpyrrolidone), synthetic and natural polymers (for example albumin), stabilizers (e.g. antioxidants such as for example ascorbic acid), colourants (e.g. inorganic pigments such as for example iron oxides) and flavour or odour correctors.

In general, to achieve effective results in parenteral administration it has been found advantageous to administer quantities of about 0.001 to 10 mg/kg, preferably about 0.01 to 1 mg/kg body weight. In oral administration, the dosage is about 0.01 bis 100 mg/kg, preferably about 0.01 to 20 mg/kg and quite especially preferably 0.1 to 15 mg/kg body weight.

Nonetheless it can sometimes be necessary to deviate from the said quantities, namely depending on body weight, administration route, individual response to the active substance, nature of the preparation and time or interval at which administration takes place. Thus in some cases it can be sufficient to manage with less than the aforesaid minimum quantity, while in other cases the stated upper limit must be exceeded. In the event of administration of larger quantities, it may be advisable to divide these into several individual administrations through the day.

The following practical examples illustrate the invention. The invention is not limited to the examples.

### EXAMPLES

### A. Experimental Methods

Advantageous pharmacological properties of the compounds of the present invention can be determined by the following methods.

The therapeutic potential of the compounds of formula (I) according to the present invention in sleep apnea can be assessed preclinically in a pig model of obstructive sleep apnea (OSA).

Using negative pressure, it is possible to induce collapse and thus obstruction of the upper respiratory tract in anaesthetized, spontaneously breathing pigs (Wirth K. J. et al., Sleep 36(5) (2013) pp. 699-708).

German Landrace pigs are used for the model. The pigs are anaesthetized and tracheotomized. Two tracheal are inserted into the trachea, one into the rostral part and the other into the caudal part of the trachea. Using a connection piece, the rostral cannula is connected to a tube to the negative pressure device and to the distal tracheal cannula. The distal tracheal cannula is additionally connected to a tube with an open end to atmosphere via a connection piece that served for free tracheal breathing, circumventing the upper airway. By appropriate opening and clamping of those tubes breathing can be switched from nasal breathing to breathing through the caudal tracheal cannula, circumventing the upper airway, and the (isolated) upper airway can be connected to the negative pressure device, causing airflow in the inspiratory direction.

At certain points in time, the collapsibility of the upper respiratory tract is tested by having the pig breathe via the caudal cannula and applying negative pressures of −50, −100 and −150 cm water head (cm H₂O) to the upper respiratory tract. This causes the upper respiratory tract to collapse, which manifests itself in an interruption of the airflow and a pressure drop in the tube system. This test is conducted prior to the administration of the test substance and at certain intervals after the administration of the test substance. An appropriately effective test substance prevents this collapse of the respiratory tract in the inspiratory phase.

In this OSA pig model, systemic application of an α2-Adrenoceptor subtype C (alpha-2C) antagonists of formula (I) with an appropriate dose, reaching IC₅₀ of the alpha-2C receptor, is expected to inhibit upper airway collapsibility.

1. Compounds of formula (I) 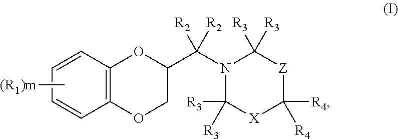
wherein
X is C(R₅)(R₆) or C(R₇)(R₈);
Z is —[C(R₄)₂]n- or a single bond;
R₁ is, independently at each occurrence, hydroxy, (C₁-C₆)alkyl, (C₁-C₆)alkoxy, halogen, halo(C₁-C₆)alkyl, phenyl(C₁-C₆)alkoxy, (C₁-C₆)alkoxy-(C═O)—, CN, NO₂, NH₂, mono- or di(C₁-C₆)alkylamino or carboxy;
R₂ is, independently at each occurrence, H or (C₁-C₆)alkyl;
R₃ is, independently at each occurrence, H or (C₁-C₆)alkyl;
R₄ is, independently at each occurrence, H, hydroxy, (C₁-C₆)alkyl, (C₂-C₆)alkenyl, hydroxy(C₁-C₆)alkyl, hydroxy(C₂-C₆)alkenyl, (C₁-C₆)alkoxy(C₁-C₆)alkyl, (C₂-C₆)alkenyloxy(C₁-C₆)alkyl, (C₁-C₆)alkyl-(C═O)—, (C₁-C₆)alkoxy-(C═O)—, hydroxy(C₁-C₆)alkoxy(C₁-C₆)alkyl or (C₁-C₆)alkoxy(C₁-C₆)alkoxy(C₁-C₆)alkyl;
or
R₄ and R₄ both attached to the same carbon ring atom form, together with the carbon ring atom to which they are attached, a —(C═O)— group;
R₅ is H or hydroxy;
or
R₄ and R₅ attached to adjacent carbon ring atoms form a bond between the carbon ring atoms to which they are attached;
R₆ is phenyl unsubstituted or substituted with 1 or 2 substituent(s) R₉;
R₇ is (C₁-C₆)alkyl, hydroxy(C₁-C₆)alkyl, (C₁-C₆)alkoxy(C₁-C₆)alkyl or (C₁-C₆)alkoxy-(C═O)—;
R₈ is hydroxy, (C₁-C₆)alkyl, (C₂-C₆)alkenyl, (C₁-C₆)alkoxy, halogen, hydroxy(C₁-C₆)alkyl, halo(C₁-C₆)alkyl, hydroxy(C₂-C₆)alkenyl, hydroxy(C₁-C₆)alkoxy, halo(C₁-C₆)alkoxy, phenyl(C₁-C₆)alkoxy, (C₁-C₆)alkoxy(C₁-C₆)alkyl, (C₂-C₆)alkenyloxy(C₁-C₆)alkyl, (C₁-C₆)alkyl-(C═O)—, (C₁,—C₆)alkoxy-(C═O)—, (C₁-C₆)alkoxy-(C═O)—(C₁-C₆)alkyl, (C₁-C₆)alkyl-S—, (C₁-C₆)alkyl-(C═O)—O—, (C₁-C₆)alkyl-(C═O)—O—(C₁-C₆)alkyl, hydroxy-(C═O)—(C₁-C₆)alkoxy, hydroxy-(C═O)—(C₁-C₆)alkoxy(C₁-C₆)alkyl, (C₁-C₆)alkoxy-(C═O)—(C₁-C₆)alkoxy(C₁-C₆)alkyl, (C₁-C₆)alkyl-S—(C₁-C₆)alkyl, hydroxy(C₁-C₆)alkyl-S—(C₁-C₆)alkyl, (C₁-C₆)alkoxy(C₁-C₆)alkyl-S—(C₁-C₆)alkyl, (C₁-C₆)alkyl-(C═O)—S—(C₁-C₆)alkyl (C₁-C₆)alkoxy-(C═O)—(C₁-C₆)alkyl-S—(C₁-C₆)alkyl, (C₁-C₆)alkyl-S—(C₁-C₆)alkoxy(C₁-C₆)alkyl, hydroxy(C₁-C₆)alkyl-S—(C₁-C₆)alkoxy(C₁-C₆)alkyl, (C₁-C₆)alkoxy(C₁-C₆)alkyl-S—(C₁-C₆)alkoxy(C₁-C₆)alkyl, (C₁-C₆)alkyl-(O═S═O)—(C₁-C₆)alkyl, hydroxy(C₁-C₆)alkyl-(O═S═O)—(C₁-C₆)alkyl, (C₁-C₆)alkoxy(C₁-C₆)alkyl-(O═S═O)—(C₁-C₆)alky, (C₁-C₆)alkyl-(O═S═O)—(C₁-C₆)alkoxy(C₁-C₆)alkyl, hydroxy(C₁-C₆)alkyl-(O═S═O)—(C₁-C₆)alkoxy(C₁-C₆)alkyl, (C₁-C₆)alkoxy(C₁-C₆)alkyl-(O═S═O)—(C₁-C₆)alkoxy(C₁-C₆)alkyl, (C₁-C₆)alkyl-O—(O═S═O)—(C₁-C₆)alkyl, hydroxy(C₁-C₆)alkyl-O—(O═S═O)—(C₁-C₆)alkyl, (C₁-C₆)alkyl-(O═S═O)—O—(C₁-C₆)alkyl, hydroxy(C₁-C₆)alkyl-(O═S═O)—O—(C₁-C₆)alkyl, (C₁-C₆)alkyl-(O═S═O)—N(R₁₀)—(C₁-C₆)alkyl, hydroxy(C₁-C₆)alkoxy(C₁-C₆)alkyl, (C₁-C₆)alkoxy(C₁-C₆)alkoxy(C₁-C₆)alkyl, halo(C₁-C₆)alkoxy(C₁-C₆)alkyl, CN, NO₂, (R₁₀)₂N—, (R₁₀)₂N—(C₁-C₆)alkyl, (R₁₀)₂N—(C═O)—, (R₁₀)₂N—(C═O)—(C₁-C₆)alkoxy(C₁-C₆)alkyl, (R₁₀)₂N—(C═O)—(C₁-C₆)alkyl-S—(C₁-C₆)alkyl, (R₁₀)₂N—(C═O)—(C₁-C₆)alkyl-(O═S═O)—(C₁-C₆)alkyl, carboxy, R₁₁—(O═S═O)—, R₁₁—(O═S═O)—O— or (C₁-C₆)alkoxy-(C₁-C₆)alkoxy;
or
R₄ and R₈ attached to adjacent carbon ring atoms form, together with the carbon ring atoms to which they are attached, a condensed phenyl ring, wherein said phenyl ring is unsubstituted or substituted with 1 or 2 substituent(s) each independently being hydroxy, (C₁-C₆)alkyl, (C₁-C₆)alkoxy, hydroxy(C₁-C₆)alkyl, halo(C₁-C₆)alkyl, (C₁-C₆)alkoxy(C₁-C₆)alkyl, (C₁-C₆)alkyl-(C═O)— or (C₁-C₆)alkoxy-(C═O)—;
or
R₄ and R₈ attached to adjacent carbon ring atoms form, together with the carbon ring atoms to which they are attached, a condensed 5 or 6 membered saturated or unsaturated carbocyclic ring or a condensed 5 or 6 membered saturated or unsaturated heterocyclic ring containing 1 or 2 heteroatom(s) selected from N, O, and S,
wherein said
carbocyclic or heterocyclic ring is unsubstituted or substituted with 1 or 2 substituent(s) each independently being hydroxy, (C₁-C₆)alkyl, (C₁-C₆)alkoxy, hydroxy(C₁-C₆)alkyl, halo(C-C₆)alkyl, (C₁-C₆)alkoxy(C₁-C₆)alkyl, (C₁-C₆)alkyl-(C═O)—, (C₁-C₆)alkoxy-(C═O)— or oxo;
or
R₇ and R₈ form, together with the carbon ring atom to which they are attached, a 5 or 6 membered saturated carbocyclic ring or a 5 or 6 membered saturated heterocyclic ring containing 1 or 2 heteroatom(s) selected from N, O, and S, wherein said carbocyclic or heterocyclic ring is unsubstituted or substituted with 1 or 2 substituent(s) each independently being hydroxy, (C₁-C₆)alkyl or oxo;
R₉ is, independently at each occurrence, hydroxy, (C₁-C₆)alkyl, (C₂-C₆)alkenyl, (C₁-C₆)alkoxy, halogen, hydroxy(C₁-C₆)alkyl, halo(C₁-C₆)alkyl, hydroxy(C₂-C₆)alkenyl, hydroxy(C₁-C₆)alkoxy, halo(C₁-C₆)alkoxy, phenyl(C₁-C₆)alkoxy, (C₁-C₆)alkoxy(C₁-C₆)alkyl, (C₂-C₆)alkenyloxy(C₁-C₆)alkyl, (C₁-C₆)alkyl-(C═O)—, (C₁-C₆)alkoxy-(C═O)—, (C₁-C₆)alkoxy-(C═O)—(C₁-C₆)alkyl, (C₁-C₆)alkyl-S—, (C₁-C₆)alkyl-(C═O)—O—, (C₁-C₆)alkyl-(C═O)—O—(C₁-C₆)alkyl, hydroxy-(C═O)—(C₁-C₆)alkoxy, hydroxy-(C═O)—(C₁-C₆)alkoxy(C₁-C₆)alkyl, (C₁-C₆)alkoxy-(C═O)—(C₁-C₆)alkoxy(C₁-C₆)alkyl, (C₁-C₆)alkyl-S—(C₁-C₆)alkyl, hydroxy(C₁-C₆)alkyl-S—(C₁-C₆)alkyl, (C₁-C₆)alkoxy(C₁-C₆)alkyl-S—(C₁-C₆)alkyl, (C₁-C₆)alkyl-(C═O)—S—(C₁-C₆)alkyl, (C₁-C₆)alkoxy-(C═O)—(C₁-C₆)alkyl-S—(C₁-C₆)alkyl, (C₁-C₆)alkyl-S—(C₁-C₆)alkoxy(C₁-C₆)alkyl, hydroxy(C₁-C₆)alkyl-S—(C₁-C₆)alkoxy(C₁-C₆)alkyl, (C₁-C₆)alkoxy(C₁-C₆)alkyl-S—(C₁-C₆)alkoxy(C₁-C₆)alkyl, (C₁-C₆)alkyl-(O═S═O)—(C₁-C₆)alkyl, hydroxy(C₁-C₆)alkyl-(O═S═O)—(C₁-C₆)alkyl, (C₁-C₆)alkoxy(C₁-C₆)alkyl-(O═S═O)—(C₁-C₆)alkyl, (C₁-C₆)alkyl-(O═S═O)—(C₁-C₆)alkoxy(C₁-C₆)alkyl, hydroxy(C₁-C₆)alkyl-(O═S═O)—(C₁-C₆)alkoxy(C₁-C₆)alkyl, (C₁-C₆)alkoxy(C₁-C₆)alkyl-(O═S═O)—(C₁-C₆)alkoxy(C₁-C₆)alkyl, (C₁-C₆)alkyl-O—(O═S═O)—(C₁-C₆)alkyl, hydroxy(C₁-C₆)alkyl-O—(O═S═O)—(C₁-C₆)alkyl, (C₁-C₆)alkyl-(O═S═O)—O—(C₁-C₆)alkyl, hydroxy(C₁-C₆)alkyl-(O═S═O)—O—(C₁-C₆)alkyl, (C₁-C₆)alkyl-(O═S═O)—N(R₁₀)—(C₁-C₆)alkyl, hydroxy(C₁-C₆)alkoxy(C₁-C₆)alkyl, (C₁-C₆)alkoxy(C₁-C₆)alkoxy(C₁-C₆)alkyl, halo(C₁-C₆)alkoxy(C₁-C₆)alkyl, CN, NO₂, (R₁₀)₂N—, (R₁₀)₂N—(C₁-C₆)alkyl, (R₁₀)₂N—(C═O)—, (R₁₀)₂N—(C═O)—(C₁-C₆)alkoxy(C₁-C₆)alkyl, (R₁₀)₂N—(C═O)—(C₁-C₆)alkyl-S—(C₁-C₆)alkyl, (R₁₀)₂N—(C═O)—(C₁-C₆)alkyl-(O═S═O)—(C₁-C₆)alkyl, carboxy, R₁₁—(O═S═O)— or R₁₁—(O═S═O)—O—;
or
R₉ and R₉ attached to adjacent carbon ring atoms form, together with the carbon ring atoms to which they are attached, a condensed phenyl ring, a condensed 5 or 6 membered unsaturated carbocyclic ring or a condensed 5 or 6 membered unsaturated heterocyclic ring containing 1 or 2 heteroatom(s) selected from O and S, wherein said phenyl, carbocyclic or heterocyclic ring is unsubstituted or substituted with 1 or 2 substituent(s) each independently being hydroxy, (C₁-C₆)alkyl, (C₁-C₆)alkoxy, hydroxy(C₁-C₆)alkyl, halo(C₁-C₆)alkyl, (C₁-C₆)alkoxy(C₁-C₆)alkyl, (C₁-C₆)alkyl-(C═O)— or (C₁-C₆)alkoxy-(C═O)—;
R₁₀ is, independently at each occurrence, H, (C₁-C₆)alkyl, hydroxy(C₁-C₆)alkyl, (C₁-C₆)alkyl-(C═O)— or R₁₁—(O═S═O)—;
R₁₁ is, independently at each occurrence, hydroxy, (C₁-C₆)alkyl, (C₁-C₆)alkoxy, halo(C₁-C₆)alkyl or mono- or di(C₁-C₆)alkylamino;
m is 0, 1 or 2; and
n is 1 or 2;
or a pharmaceutically acceptable salt or ester thereof,
with the provisos that the compound is not 1-(7-tert-butyl-2,3-dihydrobenzo[1,4]dioxin-2-ylmethyl)-3,3-dimethyl-piperidine, 1-(6-tert-butyl-2,3-dihydrobenzo[1,4]dioxin-2-ylmethyl)-3,3-dimethyl-piperidine, 2-(2,3-dihydrobenzo[1,4]dioxin-2-ylmethyl)-6,7-dimethoxy-1-methyl-1,2,3,4-tetrahydro-isoquinoline, 2-(2,3-dihydrobenzo[1,4]dioxin-2-ylmethyl)-6,7-dimethoxy-1,2,3,4-tetrahydro-isoquinoline, 2-(2,3-dihydrobenzo[1,4]dioxin-2-ylmethyl)-1,2,3,4-tetrahydroisoquinoline or 1-(2,3-dihydro-benzo[1,4]dioxin-2-ylmethyl-3-(3-trifluoromethyl-phenyl)-pyrrolidine,
wherein the compounds of formula (I) are useful for the treatment of sleep-related breathing disorders comprising
obstructive and central sleep apneas and snoring.

2. A compound according to claim 1, wherein Z is —[C(R₄)₂]n- and n is 1.

3. A compound of claim 1, wherein
R₂ is H,
R₃ is H,
R₄ is, independently at each occurrence, H, hydroxy or (C₁-C₆)alkyl.

4. A compound of claim 1, wherein
m is 0,
R₁ is hydroxy, (C₁-C₆)alkyl, (C₁-C₆)alkoxy or halogen.

5. A compound of claim 1,
wherein X is C(R₅)(R₆), and/or
wherein R₅ is H, and/or
wherein R₆ is phenyl substituted with 1 substituent R₉,
and R₉ is hydroxy, (C₁-C₆)alkyl, (C₁-C₆)alkoxy, halogen, hydroxy(C₁-C₆)alkoxy, or (R₁₀)₂N.

6. A compound of claim 1,
wherein X is C(R₇)(R₈); and/or
wherein R₇ is (C₁-C₆)alkyl, hydroxy(C₁-C₆)alkyl, (C₁-C₆)alkoxy(C₁-C₆)alkyl or (C₁-C₆)alkoxy-(C═O)—; and/or
wherein R₈ is hydroxy(C₁-C₆)alkyl, hydroxy(C₁-C₆)alkoxy, (C₁-C₆)alkoxy(C₁-C₆)alkyl or hydroxy(C₁-C₆)alkoxy(C₁-C₆)alkyl.

7. A compound according to claim 1, wherein
X is C(R₅)(R₆) or C(R₇)(R₈);
Z is —[C(R₄)₂]n- or a single bond;
R₁ is, independently at each occurrence, hydroxy, (C₁-C₆)alkyl, (C₁-C₆)alkoxy, halogen, phenyl(C₁-C₆)alkoxy or NO₂;
R₂ is, independently at each occurrence, H;
R₃ is, independently at each occurrence, H;
R₄ is, independently at each occurrence, H, hydroxy or (C₁-C₆)alkyl;
or
R₄ and R₅ both attached to the same carbon ring atom form, together with the carbon ring atom to which they are attached, a —(C═O)— group;
R₅ is H;
or
R₄ and R₅ attached to adjacent carbon ring atoms form a bond between the carbon ring atoms to which they are attached;
R₆ is phenyl unsubstituted or substituted with 1 or 2 substituent(s) R₉;
R₇ is (C₁-C₆)alkyl, hydroxy(C₁-C₆)alkyl, (C₁-C₆)alkoxy(C₁-C₆)alkyl or (C₁-C₆)alkoxy-(C═O)—;
R₈ is hydroxy, (C₁-C₆)alkyl, (C₂-C₆)alkenyl, (C₁-C₆)alkoxy, halogen, hydroxy(C₁-C₆)alkyl, halo(C₁-C₆)alkyl, hydroxy(C₁-C₆)alkoxy, (C₁-C₆)alkoxy(C₁-C₆)alkyl, (C₂-C₆)alkenyloxy(C₁-C₆)alkyl, (C₁-C₆)alkyl-(C═O)—, (C₁-C₆)alkoxy-(C═O)—, (C₁-C₆)alkoxy-(C═O)—(C₁-C₆)alkyl, (C₁-C₆)alkyl-(C═O)—O—(C₁-C₆)alkyl, hydroxy-(C═O)—(C₁-C₆)alkoxy(C₁-C₆)alkyl, (C₁-C₆)alkoxy-(C═O)—(C₁-C₆)alkoxy(C₁-C₆)alkyl, hydroxy(C₁-C₆)alkyl-S—(C₁-C₆)alkyl, (C₁-C₆)alkyl-(C═O)—S—(C₁-C₆)alkyl, (C₁-C₆)alkyl-(O═S═O)—O—(C₁-C₆)alkyl, hydroxy(C₁-C₆)alkoxy(C₁-C₆)alkyl, (C₁-C₆)alkoxy(C₁-C₆)alkoxy(C₁-C₆)alkyl, halo(C₁-C₆)alkoxy(C₁-C₆)alkyl, CN, (R₁₀)₂N—(C₁-C₆)alkyl, (R₁₀)₂N—(C═O)—, carboxy, or (C₁-C₆)alkoxy-(C₁-C₆)alkoxy;
or
R₄ and R₈ attached to adjacent carbon ring atoms form, together with the carbon ring atoms to which they are attached, a condensed phenyl ring, wherein said phenyl ring is unsubstituted or substituted with 1 or 2 substituent(s) each independently being hydroxy(C₁-C₆)alkyl or (C₁-C₆)alkoxy-(C═O)—;
or
R₄ and R₈ attached to adjacent carbon ring atoms form, together with the carbon ring atoms to which they are attached, a condensed 6 membered saturated carbocyclic ring, wherein said carbocyclic ring is unsubstituted;
R₉ is, independently at each occurrence, hydroxy, (C₁-C₆)alkyl, (ci-C₆)alkoxy, halogen, hydroxy(C₁-C₆)alkyl, halo(C₁-C₆)alkyl, hydroxy(C₁-C₆)alkoxy, halo(C₁-C₆)alkoxy, phenyl(C₁-C₆)alkoxy, (C₁-C₆)alkoxy-(C═O)—, (C₁-C₆)alkyl-(C═O)—O—, hydroxy-(C═O)—(C₁-C₆)alkoxy, CN, (R₁₀)₂N—, (R₁₀)₂N—(C═O)—, R₁₁—(O═S═O)— or R₁₁—(O═S═O)—O—;
or
R₉ and R₉ attached to adjacent carbon ring atoms form, together with the carbon ring atoms to which they are attached, a condensed 5 or 6 membered unsaturated heterocyclic ring containing 1 or 2 heteroatom(s) selected from O, wherein said phenyl, carbocyclic or heterocyclic ring is unsubstituted;
R₁₀ is, independently at each occurrence, H or (C₁-C₆)alkyl, R₁₁ is, independently at each occurrence, (C₁-C₆)alkyl or halo(C₁-C₆)alkyl;
m is 0, 1 or 2; and
n is 1 or 2.

8. A compound according to claim 7, wherein
X is C(R₅)(R₆) or C(R₇)(R₈);
Z is —[C(R₄)₂]n-;
R₁ is, independently at each occurrence, hydroxy, (C₁-C₆)alkyl, or halogen;
R₂ is, independently at each occurrence, H;
R₃ is, independently at each occurrence, H;
R₄ is, independently at each occurrence, H;
R₅ is H;
R₆ is phenyl unsubstituted or substituted with 1 or 2 substituent(s) R₉;
R₇ is (C₁-C₆)alkyl;
R₈ is hydroxy(C₁-C₆)alkyl, hydroxy(C₁-C₆)alkoxy, (C₁-C₆)alkoxy(C₁-C₆)alkyl or hydroxy(C₁-C₆)alkoxy(C₁-C₆)alkyl,
R₉ is, independently at each occurrence, hydroxy, (C₁-C₆)alkyl, (C₁-C₆)alkoxy, halogen, hydroxy(C₁-C₆)alkoxy or (R₁₀)₂N—;
R₁₀ is, independently at each occurrence, H or (C₁-C₆)alkyl,
m is 0 or 1;
n is 1.

9. A compound according to claim 7, wherein
X is C(R₅)(R₆) or C(R₇)(R₈);
Z is a single bond;
R₁ is, independently at each occurrence, hydroxy, (C₁-C₆)alkyl, or halogen;
R₂ is, independently at each occurrence, H;
R₃ is, independently at each occurrence, H;
R₄ is, independently at each occurrence, H;
R₅ is H;
R₆ is phenyl unsubstituted or substituted with 1 or 2 substituent(s) R₉;
R₇ is (C₁-C₆)alkyl;
R₈ is hydroxy(C₁-C₆)alkyl, hydroxy(C₁-C₆)alkoxy, (C₁-C₆)alkoxy(C₁-C₆)alkyl or hydroxy(C₁-C₆)alkoxy(C₁-C₆)alkyl,
R₉ is, independently at each occurrence, hydroxy, (C₁-C₆)alkyl, (C₁-C₆)alkoxy, halogen, hydroxy(C₁-C₆)alkoxy or (R₁₀)₂N—;
R₁₀ is, independently at each occurrence, H or (C₁-C₆)alkyl,
m is 0 or 1;
n is 1.

10. A compound according to claim 1, wherein the compound is 1-(2,3-dihydrobenzo[1,4]dioxin-2-ylmethyl)-5-phenyl-1,2,3,6-tetrahydropyridine, 1-(2,3-dihydrobenzo[1,4]dioxin-2-ylmethyl)-3-phenylpiperidine, 1-(2,3-dihydrobenzo[1,4]dioxin-2-ylmethyl)-3-(2-methoxyphenyl)piperidine, 3-(4-chlorophenyl)-1-(2,3-dihydrobenzo[1,4]dioxin-2-ylmethyl)piperidine, 1-(2,3-dihydrobenzo[1,4]dioxin-2-ylmethyl)-3-(3-methoxyphenyl)piperidine, 1-(2,3-dihydrobenzo[1,4]dioxin-2-ylmethyl)-3-p-tolylpiperidine, 1-(2,3-dihydrobenzo[1,4]dioxin-2-ylmethyl)-3-(4-methoxyphenyl)piperidine,1-(2,3-dihydrobenzo[1,4]dioxin-2-ylmethyl)-3-o-tolylpiperidine, 4-[1-(2,3-dihydrobenzo[1,4]dioxin-2-ylmethyl)piperidin-3-yl]phenol, 1-(2,3-dihydrobenzo[1,4]dioxin-2-ylmethyl)-3-(3-fluorophenyl)piperidine, 3-[1-(2,3-dihydrobenzo[1,4]dioxin-2-ylmethyl)piperidin-3-yl]phenol, 2-[1-(2,3-dihydrobenzo[1,4]dioxin-2-ylmethyl)piperidin-3-yl]phenol, (R*)-1-[(S)-1-(2,3-dihydrobenzo[1,4]dioxin-2-yl)methyl]-3-(2-fluorophenyl)piperidine, (S*)-1-[(S)-1-(2,3-dihydrobenzo[1,4]dioxin-2-yl)methyl]-3-(2-fluorophenyl)piperidine, (R*)-1-[(S)-1-(2,3-dihydrobenzo[1,4]dioxin-2-yl)methyl]-3-(3-fluorophenyl)piperidine, (R*)-1-[(S)-1-(2,3-dihydrobenzo[1,4]dioxin-2-yl)methyl]-3-(3-fluorophenyl)piperidine ·HCl, (S*)-1-[(S)-1-(2,3-dihydrobenzo[1,4]dioxin-2-yl)methyl]-3-(3-fluorophenyl)piperidine, (R*)-1-[(S)-1-(2,3-dihydrobenzo[1,4]dioxin-2-yl)methyl]-3-(4-fluorophenyl)piperidine, (S*)-1-[(S)-1-(2,3-dihydrobenzo[1,4]dioxin-2-yl)methyl]-3-(4-fluorophenyl)piperidine, (R*)-1-[(S)-1-(2,3-dihydrobenzo[1,4]dioxin-2-yl)methyl]-3-(3-trifluoromethyl-phenyl)piperidine, (S*)-1-[(S)-1-(2,3-dihydrobenzo[1,4]dioxin-2-yl)methyl]-3-(3-trifluoromethyl-phenyl)piperidine, (R*)-1-[(S)-1-(2,3-dihydrobenzo[1,4]dioxin-2-yl)methyl]-3-(4-trifluoromethylphenyl)piperidine, (S*)-1-[(S)-1-(2,3-dihydrobenzo[1,4]dioxin-2-yl)methyl]-3-(4-trifluoromethylphenyl)piperidine, (R*)-1-[(S)-1-(2,3-dihydrobenzo[1,4]dioxin-2-yl)methyl]-3-(3-methoxyphenyl)piperidine, (R*)-1-[(S)-1-(2,3-dihydrobenzo[1,4]dioxin-2-yl)methyl]-3-(3-methoxyphenyl)piperidine ·HCl, (S*)-1-[(S)-1-(2,3-dihydrobenzo[1,4]dioxin-2-yl)methyl]-3-(3-methoxyphenyl)piperidine, (R*)-3-{1-[(S)-1-(2,3-dihydrobenzo[1,4]dioxin-2-yl)methyl]piperidin-3-yl}phenol, (R*)-3-{1-[(S)-1-(2,3-dihydrobenzo[1,4]dioxin-2-yl)methyl]piperidin-3-yl}phenol HCl, (3-{(R*)-1-[(S)-1-(2,3-dihydrobenzo[1,4]dioxin-2-yl)methyl]piperidin-3-1}phenyl)methanol, acetic acid 3-{(S)-1-[(R*)-1-(2,3-dihydrobenzo[1,4]dioxin-2-yl)methyl]piperidin-3-yl}phenyl ester ·HCl, 2-(3-{(R*)-1-[(S)-1-(2,3-dihydrobenzo[1,4]dioxin-2-yl)methyl]piperidin-3-yl}phenoxy)ethanol, 3-(3-{(R*)-1-[(S)-1-(2,3-dihydrobenzo[1,4]dioxin-2-yl)methyl]piperidin-3-yl}phenoxy)propan-1-ol, trifluoromethanesulfonic acid 3-{(R*)-1-[(S)-1-(2,3-dihydrobenzo[1,4]dioxin-2-yl)methyl]-piperidin-3-yl}phenyl ester, (3-{(R*)-1-[(S)-1-(2,3-dihydrobenzo[1,4]dioxin-2-yl)methyl]piperidin-3-yl}phenoxy)acetic acid, 3-{(R*)-1-[(S)-1-(2,3-dihydrobenzo[1,4]dioxin-2-yl)methyl]-piperidin-3-yl}-benzonitrile, 3-benzo[1,3]dioxol-5-yl-1-[(S)-1-(2,3-dihydrobenzo[1,4]dioxin-2-yl)methyl]-piperidine, 3-[(R*)-1-((S)-6-benzyloxy-2,3-dihydro-benzo[1,4]dioxin-2-ylmethyl)-piperidin-3-yl]-phenol, (S)-2-[(R*)-3-(3-hydroxy-phenyl)-piperidin-1-ylmethyl]-2,3-dihydro-benzo[1,4]dioxin-6-ol, 3-[(R*)-1-((S)-7-benzyloxy-2,3-dihydro-benzo[1,4]dioxin-2-ylmethyl)-piperidin-3-yl]-phenol, (S)-3-[(R*)-3-(3-hydroxyphenyl)-piperidin-1-ylmethyl]-2,3-dihydro-benzo[1,4]dioxin-6-ol, 3-[(R*)-1-((S)-8-benzyloxy-2,3-dihydrobenzo[1,4]dioxin-2-ylmethyl)piperidin-3-yl]phenol, (S)-3-[(R*)-3-(3-hydroxy-phenyl)-piperidin-1-ylmethyl]-2,3-dihydrobenzo[1,4]dioxin-5-ol, (R*)-3-[1-((S)-7-fluoro-2,3-dihydrobenzo[1,4]dioxin-2-ylmethyl)piperidin-3-yl]phenol, 1-(2,3-dihydrobenzo[1,4]dioxin-2-ylmethyl)-3-methylpiperidine-3-carboxylic acid ethyl ester, 1-(2,3-dihydrobenzo[1,4]dioxin-2-ylmethyl)-3-ethylpiperidine-3-carboxylic acid ethyl ester, [1-(2,3-dihydrobenzo[1,4]dioxin-2-ylmethyl)-3-methylpiperidin-3-yl]methanol, 1-(2,3-dihydrobenzo[1,4]dioxin-2-ylmethyl)-3-methoxymethyl-3-methylpiperidine, 1-(2,3-dihydrobenzo[1,4]dioxin-2-ylmethyl)-3-ethoxymethyl-3-methylpiperidine, 3-chloromethyl-1-(2,3-dihydrobenzo[1,4]dioxin-2-ylmethyl)-3-methylpiperidine, 2-[1-(2,3-dihydrobenzo[1,4]dioxin-2-ylmethyl)-3-methylpiperidin-3-yl]propan-2-ol, 1-(2,3-dihydrobenzo[1,4]dioxin-2-ylmethyl)-3-(1-methoxy-1-methylethyl)-3-methylpiperidine, 1-(2,3-dihydrobenzo[1,4]dioxin-2-ylmethyl)-3-hydroxymethyl-3-methylpiperidin-4-ol, acetic acid 1-(2,3dihydrobenzo[1,4]dioxin-2-ylmethyl)-3-methylpiperidin-3-ylmethyl ester, methanesulfonic acid 1-(2,3-dihydrobenzo[1,4]dioxin-2-ylmethyl)-3-methylpiperidin-3-ylmethyl ester, [1-(2,3-dihydrobenzo[1,4]dioxin-2-ylmethyl)-3-ethylpiperidin-3-yl]methanol, 1-(2,3-dihydrobenzo[1,4]dioxin-2-ylmethyl)-3-ethyl-3-methoxymethylpiperidine, 1-(2,3-dihydrobenzo[1,4]dioxin-2-ylmethyl)-3methoxymethoxymethyl-3-methylpiperidine, 1-[1-(2,3-dihydrobenzo[1,4]dioxin-2-ylmethyl)-3-ethylpiperidin-3-yl]ethanone, 1-[1-(2,3-dihydrobenzo[1,4]dioxin-2-ylmethyl)-3-ethylpiperidin-3-yl]ethanol, 3-allyloxymethyl-1-(2,3-dihydrobenzo[1,4]dioxin-2-ylmethyl)-3-ethylpiperidine, 2-[1-(2,3-dihydrobenzo[1,4]dioxin-2-ylmethyl)-3-ethylpiperidin-3-yl-methoxy]ethanol, 3-allyloxymethyl-1-(2,3-dihydrobenzo[1,4]dioxin-2-ylmethyl)-3-methyl-piperidine, 3-allyl-1-(2,3-dihydrobenzo[1,4]dioxin-2-ylmethyl)-piperidine-3-carboxylic acid ethyl ester, [3-allyl-1-(2,3-dihydrobenzo[1,4]dioxin-2-ylmethyl)piperidin-3-yl]methanol, 3-allyl-1-(2,3-dihydrobenzo[1,4]dioxin-2-ylmethyl)-3-methoxymethylpiperidine, 3-allyl-1-(2,3-dihydrobenzo[1,4]dioxin-2-ylmethyl)-3-ethoxymethylpiperidine, 1-(2,3-dihydrobenzo[1,4]dioxin-2-ylmethyl)-3-methyl-3-(2,2,2-trifluoroethoxymethyl)-piperidine, 2-[1-(2,3-dihydrobenzo[1,4]dioxin-2-ylmethyl)-3-methylpiperidin-3-ylmethoxy]ethanol, (S)-1-((R)-2,3-dihydrobenzo[1,4]dioxine-2-carbonyl)-3-methylpiperidine-3-carboxylic acid ethyl ester, lithium (S)-1-[(S)-1-(2,3-dihydrobenzo[1,4]dioxin-2-yl)methyl]-3-methyl-piperidine-3-carboxylate, {(S)-1-[(S)-1-(2,3-dihydrobenzo[1,4]dioxin-2-yl)methyl]-3-methylpiperidin-3-yl}methanol, 2-{(S)-1-[(S)-1-(2,3-dihydrobenzo[1,4]dioxin-2-yl)methyl]-3-methylpiperidin-3-ylmethoxy}-ethanol, 2-{(S)-1-[(S)-1-(2,3-dihydrobenzo[1,4]dioxin-2-yl)methyl]-3-methylpiperidin-3-ylmethoxy}-ethanol D-tartrate, (S)-1-[(S)-1-(2,3-dihydrobenzo[1,4]dioxin-2-yl)methyl]-3-(2-methoxyethoxymethyl)-3-methylpiperidine, (S)-1-[(S)-1-(2,3-dihydrobenzo[1,4]dioxin-2-yl)methyl]-3-methyl-3-(2,2,2-trifluoroethoxymethyl)piperidine, methanesulfonic acid (S)-1-[(S)-1-(2,3-dihydrobenzo[1,4]dioxin-2-yl)methyl]-3-methyl-piperidin-3-ylmethyl ester, thioacetic acid S—{(S)-1-[(S)-1-(2,3-dihydrobenzo[1,4]dioxin-2-yl)methyl]-3-methylpiperidin-3-ylmethyl}ester, 2-{(S)-1-[(S)-1-(2,3-dihydrobenzo[1,4]dioxin-2-yl)methyl]-3-methylpiperidin-3-ylmethyl-sulfanyl}ethanol, {(S)-1-[(S)-1-(2,3-dihydrobenzo[1,4]dioxin-2-yl)methyl]-3-methylpiperidin-3-ylmethoxy}acetic acid tert-butyl ester, sodium {(S)-1-[(S)-1-(2,3-dihydrobenzo[1,4]dioxin-2-yl)methyl]-3-methylpiperidin-3-yl-methoxy}acetate, 2-[(S)-1-((S)-7-fluoro-2,3-dihydrobenzo[1,4]dioxin-2-ylmethyl)-3-methylpiperidin-3-yl-methoxy]ethanol, 1-(2,3-dihydrobenzo[1,4]dioxin-2-ylmethyl)-3-fluoro-piperidine-3-carboxylic acid ethyl ester,[1-(2,3-dihydrobenzo[1,4]dioxin-2-ylmethyl)-3-fluoro-piperidin-3-yl]methanol, (S)-1-[(S)-1-(2,3-dihydro-benzo[1,4]dioxin-2-yl)methyl]-3-methyl-piperidine-3-carboxylic acid ethyl ester, [(S)-1-((S)-7-fluoro-2,3-dihydro-benzo[1,4]dioxin-2-ylmethyl)-3-methyl-piperidin-3-yl]-methanol, (S)-1-((S)-7-fluoro-2,3-dihydro-benzo[1,4]dioxin-2-ylmethyl)-3-methoxymethyl-3-methyl-piperidine, (S)-1-((S)-7-fluoro-2,3-dihydro-benzo[1,4]dioxin-2-ylmethyl)-3-methoxymethyl-3-methyl-piperidine-HCl, (S)-1-[(S)-1-(2,3-dihydro-benzo[1,4]-dioxin-2-yl)methyl]-3-methoxymethyl-3-methyl-piperidine, 3-{(R*)-1-[(S)-1-(2,3-dihydro-benzo[1,4]dioxin-2-yl)methyl]-piperidin-3-yl}-phenylamine, (R*)-3-{1-1[(S)-1-(2,3-dihydrobenzo[1,4]dioxin-2-yl)methyl]piperidin-3-yl}phenol oxalate, (S)-2-[(R*)-3-(3-hydroxy-phenyl)-piperidin-1-ylmethyl]-2,3-dihydro-benzo[1,4]dioxin-5-ol, 1-[(S)-1-(2,3-dihydro-benzo[1,4]dioxin-2-yl)methyl]-3-(2-methoxy-phenyl)-pyrrolidine, (S)-1-[(S)-1-(2,3-dihydro-benzo[1,4]dioxin-2-yl)methyl]-3-(2-fluoro-ethoxymethyl)-3-methyl-piperidine ·HCl, (R*)-1-[(S)-1-(2,3-dihydro-benzo[1,4]dioxin-2-yl)methyl]-3-(3-fluoromethoxy-phenyl)-piperidine, 1-(2,3-dihydro-benzo[1,4]dioxin-2-ylmethyl)-3-methyl-pyrrolidine-3-carboxylic acid methyl ester, [1-(2,3-dihydro-benzo[1,4]dioxin-2-ylmethyl)-3-methylpyrrolidin-3-yl]-methanol, 2-[1-(2,3-dihydro-benzo[1,4]dioxin-2-ylmethyl)-3-methyl-pyrrolidin-3-ylmethoxy]-ethanol, 1-(2,3-dihydro-benzo[1,4]dioxin-2-ylmethyl)-3-methoxymethyl-3-methyl-pyrrolidine or 3-[(R)-1-((S)-7-nitro-2,3-dihydrobenzo[1,4]dioxin-2-ylmethyl)-piperidin-3-yl]-phenol.

11. A compound according to claim 1, wherein the compound is selected from the group consisting of:
(R*)-1-[(S)-1-(2,3-dihydrobenzo[1,4]dioxin-2-yl)methyl]-3-(3-fluorophenyl)piperidine, (R*)-1-[(S)-1-(2,3-dihydrobenzo[1,4]dioxin-2-yl)methyl]-3-(3-methoxyphenyl)piperidine,
(R*)-3-{1-[(S)-1-(2,3-dihydrobenzo[1,4]dioxin-2-yl)methyl]piperidin-3-yl}phenol, (3-{(R*)-1-[(S)-1-(2,3-dihydrobenzo[1,4]dioxin-2-yl)methyl]piperidin-3-yl}phenyl)methanol,
2-(3-{(R*)-1-[(S)-1-(2,3-dihydrobenzo[1,4]dioxin-2-yl)methyl]piperidin-3-yl}phenoxy)ethanol,
3-(3-{(R*)-1-[(S)-1-(2,3-dihydrobenzo[1,4]dioxin-2-yl)methyl]piperidin-3-yl}phenoxy)propan-1-ol,
(R*)-3-[1-((S)-7-fluoro-2,3-dihydrobenzo[1,4]dioxin-2-ylmethyl)piperidin-3-yl]phenol, {(S)-1-[(S)-1-(2,3-dihydrobenzo[1,4]dioxin-2-yl)methyl]-3-methylpiperidin-3-yl}methanol,
2-{(S)-1-[(S)-1-(2,3-dihydrobenzo[1,4]dioxin-2-yl)methyl]-3-methylpiperidin-3-ylmethoxy}-ethanol,
(S)-1-[(S)-1-(2,3-dihydrobenzo[1,4]dioxin-2-yl)methyl]-3-(2-methoxyethoxymethyl)-3-methylpiperidine,
and a pharmaceutically acceptable salt or ester thereof.

12.-22. (canceled)

23. A pharmaceutical composition comprising:
at least one compound of claim 1; and one or more inert non-toxic pharmaceutically suitable excipients.

24. (canceled)

25. A method for the treatment of sleep-related breathing disorders, comprising the step of administering systemically and/or locally a therapeutically effective amount of at least one compound of claim 1.

26. (canceled)

27. A medicament, comprising a compound of claim 1 and one or more further active ingredients selected from the group consisting of muscarinic receptor antagonists, mineralocorticoid receptor antagonists, diuretics, corticosteroids.
